SEQUENCE LISTING (10 sequences)

SEQ ID NO: 1 - DNA, 25 bp, Rattus norvegicus
  source 1..25: mol_type = genomic DNA
tccaaggagtataagcagagggcca

SEQ ID NO: 2 - DNA, 24 bp, Rattus norvegicus
  source 1..24: mol_type = genomic DNA
ctcttagggtctaggactagcttg

SEQ ID NO: 3 - DNA, 20 bp, Rattus norvegicus
  source 1..20: mol_type = genomic DNA
agcccagaacatcatccctg

SEQ ID NO: 4 - DNA, 21 bp, Rattus norvegicus
  source 1..21: mol_type = genomic DNA
caccaccttcttgatgtcatc

SEQ ID NO: 5 - DNA, 19 bp, Rattus norvegicus
  source 1..19: mol_type = genomic DNA
gaaccaagaaggcacagac

SEQ ID NO: 6 - DNA, 19 bp, Rattus norvegicus
  source 1..19: mol_type = genomic DNA
aatgcgccctaaatcactg

SEQ ID NO: 7 - DNA, 19 bp, Rattus norvegicus
  source 1..19: mol_type = genomic DNA
caagatggtcgccctggac

SEQ ID NO: 8 - DNA, 20 bp, Rattus norvegicus
  source 1..20: mol_type = genomic DNA
ccagggaatcccatcacacc

SEQ ID NO: 9 - DNA, 20 bp, Rattus norvegicus
  source 1..20: mol_type = genomic DNA
ctatctgggaggggtcttcc

SEQ ID NO: 10 - DNA, 20 bp, Rattus norvegicus
  source 1..20: mol_type = genomic DNA
ggttgagggtgtctgaagga

(19) United States
(12) Patent Application Publication
(10) Pub. No.: US 2026/0144849 A1
(43) Pub. Date: May 28, 2026

(54) MEMBRANE FOR GUIDED BONE REGENERATION AND METHOD FOR MANUFACTURING THE SAME

(71) Applicant: Y-BON BIO INC., Seoul (KR)

(72) Inventors: YOUNGBUM PARK, Seoul (KR); JIANPING HUANG, Seoul (KR); JAEHAN PARK, Seoul (KR); NARAE JUNG, Seoul (KR); JINGYU HE, Seoul (KR)

(21) Appl. No.: 19/401,726

(22) Filed: Nov. 26, 2025

(30) Foreign Application Priority Data
Nov. 28, 2024 (KR) 10-2024-0173921
Nov. 26, 2025 (KR) 10-2025-0182492

(51) Int. Cl. A61K 38/18; A61K 9/06; A61K 47/36; A61K 47/42; A61P 19/08

(52) U.S. Cl. CPC A61K 38/1875; A61K 9/06; A61K 47/36; A61K 47/42; A61P 19/08

(57) ABSTRACT

A membrane for guided bone regeneration includes a hydrogel containing alginate and gelatin at a weight ratio of 1:1.0-1.6, and impregnated with a bone morphogenetic protein (BMP). With this method, the membrane can be conveniently administered by syringe injection, and the manufacturing process is simple, requires no complex equipment, and is advantageous in terms of time and cost, while providing improved therapeutic effects.

### CROSS-REFERENCE TO RELATED APPLICATIONS AND CLAIM OF PRIORITY

This application claims the benefit under 35 USC § 119 of Korean Patent Application Nos. 10-2024-0173921 filed on Nov. 28, 2024 and 10-2025-0182492 filed Nov. 26, 2025 in the Korean Intellectual Property Office, the entire disclosure of which are incorporated herein by reference for all purposes.

### REFERENCE TO AN ELECTRONIC SEQUENCE LISTING

A sequence listing electronically submitted on Nov. 26, 2025 as an XML file named 20251126 LC1272521_TU_SEQ.XML, created on Nov. 26, 2025 and having a size of 9,904 bytes, is incorporated herein by reference in its entirety.

### BACKGROUND

### 1. Field of the Invention

The present invention relates to a membrane containing BMP-2 or other growth factors for rapid Guided Bone Regeneration (rGBR) to enhance rapid outer bone wall formation (rOBW).

### 2. Description of the Related Art

With the aging population, the number of patients suffering from dental problems such as loss of tooth function or alveolar bone loss has been increasing.

In the case where only teeth are lost, dental function may be restored by implanting an artificial tooth. However, in the case where alveolar bone is also lost, bone grafting is required to form new alveolar bone before implanting the artificial tooth.

Guided bone regeneration (GBR) is a common dental procedure performed to reconstruct alveolar bone loss or defects in dental practice.

The guided bone regeneration (GBR) uses a membrane to form a physical barrier between the bone defect site and surrounding soft tissues, thereby preventing soft tissue growth and providing an environment conducive to bone cell growth and regeneration.

Collagen membranes are widely used in guided bone regeneration due to their excellent biocompatibility and biodegradability. In particular, these membranes are utilized as a carrier for bone morphogenetic protein-2 (BMP-2) to effectively induce new bone formation.

However, the collagen membranes are expensive and, when implanted alone, do not provide sufficient space-maintaining function. In addition, when BMP-2 is used to induce bone formation, it is impregnated into a filler material for filling the bone defect site, rather than the collagen membrane. The use of high concentrations of BMP-2 in the bone defect site may cause various side effects. Therefore, the development of a membrane for guided bone regeneration that overcomes these problems is required.

### SUMMARY

It is an object of the present invention to provide a membrane for guided bone regeneration and a method for manufacturing the same, which not only address the problems of conventional membranes for guided bone regeneration, including causing an inflammatory response before bone formation is completely achieved, failing to provide sufficient space-maintaining function, and being expensive, but also improve therapeutic effects, reduce medical costs, and enable commercialization as an injectable or 3D-printing bioink.
1. A membrane for guided bone regeneration including a hydrogel including alginate and gelatin at a weight ratio of 1:1.0-1.6, and impregnated with a bone morphogenetic protein (BMP).
2. The membrane for guided bone regeneration according to the above 1, wherein the alginate is included at a concentration of 7 to 10% (w/v).
3. The membrane for guided bone regeneration according to the above 1, wherein the bone morphogenetic protein is included at a concentration of 0.15 to 0.35% (w/v).
4. The membrane for guided bone regeneration according to the above 1, wherein the bone morphogenetic protein is any one selected from the group consisting of BMP-2, BMP-4, BMP-6, BMP-7, and BMP-9.
5. A dual-syringe mixer including: a first syringe filled with a hydrogel preparation powder including alginate and gelatin at a weight ratio of 1:1.0-1.6; and a second syringe filled with a bone morphogenetic protein (BMP) solution.
6. The dual-syringe mixer according to the above 5, wherein the bone morphogenetic protein is included at a concentration of 0.15 to 0.35% (w/v).
7. The dual-syringe mixer according to the above 5, wherein the bone morphogenetic protein is any one selected from the group consisting of BMP-2, BMP-4, BMP-6, BMP-7, and BMP-9.
8. A method for manufacturing a membrane for guided bone regeneration, including: preparing a dispersion by dispersing alginate and gelatin in a buffer at a weight ratio of 1:1.0-1.6; preparing a mixture by mixing a bone morphogenetic protein (BMP) into the dispersion; and forming the mixture using a syringe or a 3D printer.
9. The method for manufacturing a membrane for guided bone regeneration according to the above 8, wherein the forming step is performed by injection or 3D printing using a 23 G to 25 G needle.
10. A method for promoting bone formation, including: filling a bone defect site with a filler material; and covering an upper surface of the bone defect site with a membrane for guided bone regeneration to isolate the bone defect site from the outside.
11. The method for promoting bone formation according to claim 10, wherein isolating the bone defect site from the outside includes: mixing a hydrogel preparation powder and a bone morphogenetic protein (BMP) solution using the dual-syringe mixer according to any one of claims 5 to 7; and injecting a membrane composition for promoting bone formation into the bone defect of a patient using either the first syringe or the second syringe of the dual-syringe mixer.

The present invention provides a membrane for guided bone regeneration having minimal side effects, such as inflammation, and a method for manufacturing the same.

The present invention provides a membrane for guided bone regeneration and a method for manufacturing the same, which improve the convenience of the procedure.

The method for manufacturing the membrane for guided bone regeneration of the present invention is simple, requires no complex equipment, and is advantageous in terms of time and cost.

The present invention provides a membrane for guided bone regeneration having a strength suitable for the procedure and a method for manufacturing the same, thereby improving the convenience of dental procedures.

The present invention provides a membrane for guided bone regeneration that can be conveniently used by injection with a syringe and a method for manufacturing the same.

The present invention enables the manufacturing of a membrane for guided bone regeneration that can be custom-produced and applied using a 3D printer.

### BRIEF DESCRIPTION OF THE DRAWINGS

FIG. 1 illustrates the manufacturing process of a membrane for guided bone regeneration and an analytical experimental protocol thereof.

FIGS. 2 and 3 are schematic diagrams of exemplary bone graft materials to which the membrane for guided bone regeneration manufactured according to the present invention is applied.

FIG. 4 illustrates an exemplary process for manufacturing a membrane for guided bone regeneration using 3D printing.

FIG. 5A illustrates 3A5 G, 5A5 G, 6A10 G, 8A10 G, and 4A15 G hydrogels formed on a glass cover slip without curing.

FIG. 5B illustrates 3A5 G, 5A5 G, 6A10 G, 8A10 G, and 4A15 G hydrogels formed on a glass cover slip after curing with CaCl₂) for 15 minutes.

FIGS. 6A and 6B show the appearance and water resistance test results of the struts injected with 6A10 G and 5A5 G hydrogels using a 23 G needle in a 1 mL syringe, respectively; the photos were taken immediately after injection, 5 minutes after injection, and after immersion in distilled water or after being lifted up from water and flushed with water.

FIGS. 7A to 7E show the gelation kinetics of 3A5 G, 5A5 G, 6A10 G, 8A10 G, and 4A15 G hydrogels, respectively, showing the storage modulus (G′) and loss modulus (G″) after transfer from 37° C. incubation to a 25° C. test platform.

FIGS. 8A to 8D show the sol-gel transition kinetics changes observed during the transition from high to low shear strain of 5A5 G, 8A10 G, 6A10 G, and 4A15 G hydrogels at 25° C.

FIGS. 9A to 9C illustrate the 3D-printing results of 6A10 G and 4A15 G hydrogels. FIG. 9A shows 6A10 G and 4A15 G hydrogels formed by 3D printing before and after CaCl₂) curing. FIG. 9B shows results of the product yield measured as the volume of the printed hydrogel relative to the total volume of the 6A10 G or 4A15 G solution. FIG. 9C shows the resting time window, which refers to the resting time at room temperature until the last printed sample becomes gelled during the 3D printing of the 6A10 G or 4A15 G solution (*p<0.05, **p<0.01, ***p<0.001).

FIGS. 10A and 10B illustrate the volume changes of 4A15 G and 6A10 G hydrogels measured on days 0, 1, and 2 after incubation in PBS under condition of 37° C., either uncured or cured with 2% CaCl₂) for 15 minutes; data are expressed as means and standard deviations.

FIGS. 11A and 11B illustrate the degradation kinetics of 4A15 G and 6A10 G hydrogels in culture plates; data are expressed as means and standard deviations. (*P<0.05, vs. 4A5 G hydrogel)

FIGS. 12A and 12B illustrate the degradation kinetics of 4A15 G and 6A10 G hydrogels in tubes with caps; data are expressed as means and standard deviations. (*P<0.05, vs. 4A5 G hydrogel).

FIGS. 13A and 13B illustrate the FITC-BSA release profile (FIG. 13A) and remaining FITC-BSA mass (FIG. 13B) in 6A10 G hydrogel, as determined by measuring the fluorescent FITC density on days 1, 3, 5, 7, 14, 21, and 28 under condition of 37° C.; data are expressed as means and standard deviations.

FIGS. 14A and 14B illustrate the rhBMP-2 release profile (FIG. 14A) and remaining rhBMP-2 mass (FIG. 14B) in 6A10 G hydrogels, as determined by measuring the fluorescent FITC density on days 1, 3, 5, 7, 14, 21, and 28 under condition of 37° C.; data are expressed as means and standard deviations.

FIGS. 15A and 15B illustrate the release profiles of FITC-BSA at various concentrations (0.5, 0.4, 0.3, 0.2, and 0.1 mg/mL) impregnated into 6A10 G hydrogels, as determined by measuring the fluorescent FITC density on days 1, 3, 6, 8, 14, and 21 at 37° C. In FIG. 15A, the data represent the cumulative amount (mg) of FITC-BSA released from 1 mL of the hydrogel, and FIG. 15B represents the cumulative percentage (%) of FITC-BSA released from the hydrogel; data are expressed as means and standard deviations.

FIG. 16 illustrates the results of determining the cell viability and proliferation of MSCs seeded on 6A10 G and GelMA hydrogels after 24 and 48 hours.

FIG. 17 illustrates the expression of osteogenic markers in MSCs co-cultured with rhBMP-2-impregnated 6A10 G composite hydrogels for 3 days; data are expressed as means and standard deviations. (OIM: osteogenic induction medium; B0.1:0.1 mg/mL rhBMP-2 in hydrogel, B0.25:0.25 mg/mL BMP-2 in hydrogel, B0.5:0.5 mg/mL mhBMP-2 in hydrogel; *p<0.05, **p<0.01, ***p<0.001, ****p<0.0001).

FIG. 18 illustrates the alkaline phosphatase (ALP) staining results of MSCs co-cultured with 6A10 G composite hydrogels impregnated with rhBMP-2 for 7 days.

FIG. 19 is a graph quantitatively illustrating the ALP staining results of MSCs co-cultured with 6A10 G composite hydrogels impregnated with rhBMP-2 for 7 days; data are expressed as means and standard deviations (*p<0.05, **p<0.01, ***p<0.001, ****p<0.0001).

FIG. 20 illustrates the ALP staining results of MSCs co-cultured with 6A10 G composite hydrogels impregnated with various concentrations of rhBMP-2 for 7 days.

FIG. 21 is a graph quantitatively illustrating the ALP staining results of MSCs co-cultured with 6A10 G composite hydrogels impregnated with rhBMP-2 at various concentrations (0, 0.1, 0.2, 0.3, 0.4, and 0.5 mg/mL) for 7 days; data are expressed as means and standard deviations (*p<0.05, **p<0.01, ***p<0.001, ****p<0.0001).

FIG. 22 illustrates the ARS results of MSCs co-cultured with 6A10 G composite hydrogels impregnated with rhBMP-2 for 21 days.

FIG. 23 is a graph quantitatively illustrating the ARS results of MSCs co-cultured with 6A10 G composite hydrogels impregnated with rhBMP-2 for 21 days; data are expressed as means and standard deviations (****P<0.0001).

FIG. 24 illustrates the ARS results of MSCs co-cultured with 6A10 G composite hydrogels impregnated with various concentrations of rhBMP-2 (0, 0.1, 0.2, 0.3, 0.4, and 0.5 mg/mL) for 21 days.

FIG. 25 is a graph quantitatively illustrating the ARS results of MSCs co-cultured with 6A10 G composite hydrogels impregnated with various concentrations of rhBMP-2 (0, 0.1, 0.2, 0.3, 0.4, and 0.5 mg/mL) for 21 days; data are expressed as means and standard deviations (*P<0.05, **P<0.01, ***p<0.001).

FIG. 26 illustrates the procedure of guided bone regeneration for calvarial defect in a rat.

FIG. 27 illustrates 3D images of the calvarial defect grafting results. The defect site (8 mm in diameter) in rats treated with β-TCP and collagen membrane was considered Con GBR (n=5); the defect site in rats treated with functional hydrogel loaded with β-TCP, collagen membrane, and 0.1 mg/ml mhBMP-2 was considered Exp GBR (n=5); and the rats that received no treatment were considered Blank (n=5).

FIGS. 28A and 28B illustrate the quantitative analysis of the calvarial defect graft. FIG. 28A shows the bone volume/tissue volume (BV/TV) ratio after 3 weeks of treatment, and FIG. 28B shows the BV/TV ratio after 6 weeks of treatment; data are presented as means and standard deviations (n=5 per group per time point; *p<0.05, **p<0.01, ***p<0.001, ****p<0.0001).

FIG. 29 illustrates histological images of the calvarial defect graft observed at low magnification. The area between the two green dotted lines represents the 8 mm calvarial defect site (Scale bar: 1 mm).

FIG. 30 illustrates histological images of the calvarial defect graft observed at 3 weeks after grafting at high magnification; arrows point to the β-TCP pellets (Scale bar: 200 μm).

FIG. 31 illustrates the histological results of the calvarial defect graft observed at 6 weeks after grafting at high magnification (Scale bar. 200 μm).

FIG. 32 is a graph cumulatively illustrating the percentages of new bone, β-TCP pellets, and fibrous tissue in the calvarial defect site observed at 3 and 6 weeks after grafting; data are expressed as mean percentages (n=5 per time point).

FIG. 33 illustrates the ratio of new bone area to tissue area observed in the calvarial defect site observed at 3 or 6 weeks after grafting; data are expressed as means and standard deviations (n=5 per group per time point; *p<0.05, **p<0.01, ***p<0.001).

FIG. 34 illustrates immunohistochemical staining results for osteocalcin (OCN) in the calvarial defect site at 3 or 6 weeks after grafting (Scale bar: 100 μm).

FIG. 35 illustrates quantitative analysis results of OCN expression in the region of interest (ROI); data are expressed as means and standard deviations (n=5 per group per time point; *p<0.05, **p<0.01, ***p<0.001, ****p<0.0001).

FIG. 36 illustrates the results of immunohistochemical staining for CD31 in the calvarial defect site at 3 or 6 weeks after grafting; red arrows indicate blood vessels in the ROI (Scale bar: 100 μm).

FIG. 37 illustrates the results of determining the vascular density in the bone defect site after grafting; data are expressed as means and standard deviations (n=6 per group per time point; *p<0.05, **p<0.01, ***p<0.001, ****p<0.0001).

FIG. 38 illustrates the appearance of the hydrogel after mixing with increasing gelatin content in fixed alginate.

FIG. 39 illustrates the appearance of the hydrogel after mixing with increasing alginate content in the control (6A10 G) and fixed gelatin.

FIG. 40 illustrates the compression test results for five 6A10 G specimens.

FIG. 41 illustrates the compression test results for five 8A12 G specimens.

FIG. 42 illustrates a comparison of the average values in the compression tests for 6A10 G and 8A12 G.

FIGS. 43A and 43B illustrate the tensile test results and the average values for five 8A12 G specimens.

FIGS. 44A to 44C illustrate the viscosity test results for five specimens having thicknesses of 1 mm, 2 mm, and 3 mm.

FIG. 45 illustrates the results of a two-way ANOVA for viscosity as a function of specimen thickness and shear rate.

FIGS. 46A to 46C illustrate the rheological test results showing the storage modulus (G′) and loss modulus (G″) for five specimens having thicknesses of 1 mm, 2 mm, and 3 mm.

FIG. 47 illustrates the results of a two-way ANOVA for storage modulus (G′) and loss modulus (G″) as a function of specimen thickness.

FIGS. 48A to 48D illustrates CT and 3D images of the rabbit calvarial defect model experiment results. FIGS. 48A to 48D show the results at 2, 4, 6, and 8 weeks after grafting, respectively. C represents the group in which only 6A10 G hydrogel was injected into the scaffold; SP represents the group in which 6A10 G hydrogel was injected into the scaffold and pre-eroded in PDRN solution before grafting; iB represents the group in which 6A10 G hydrogel was injected into the scaffold and BMP-2 solution was drop-injected after grafting; SB represents the group in which 6A10 G hydrogel was injected into the scaffold and pre-eroded in BMP-2 solution before grafting; AB represents the group in which BMP-2-impregnated 6A10 G hydrogel, prepared by directly adding BMP-2 to PBS during the 6A10 G hydrogel manufacturing process, was grafted.

FIGS. 49A and 49B illustrate the CT results of a rabbit calvarial defect model experiment, analyzed using the Kruskal-Wallis test.

FIG. 50 illustrates the H&E staining results of cross-sections of the calvarial defect sites in the control (C), iB, SB, SP, and AB groups at 2, 4, 6, and 8 weeks after grafting.

FIGS. 51A and 51B illustrate the results of a histological analysis of a rabbit calvarial defect model, analyzed using the Kruskal-Wallis test.

FIG. 52 illustrates the results of a two-way ANOVA of the histological analysis of a rabbit calvarial defect model.

FIG. 53 illustrates the dual-syringe mixer of the present invention.

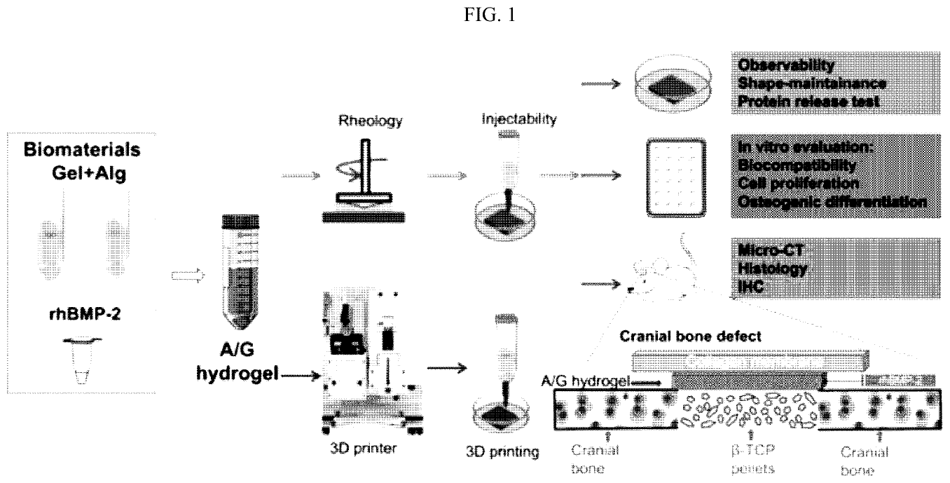

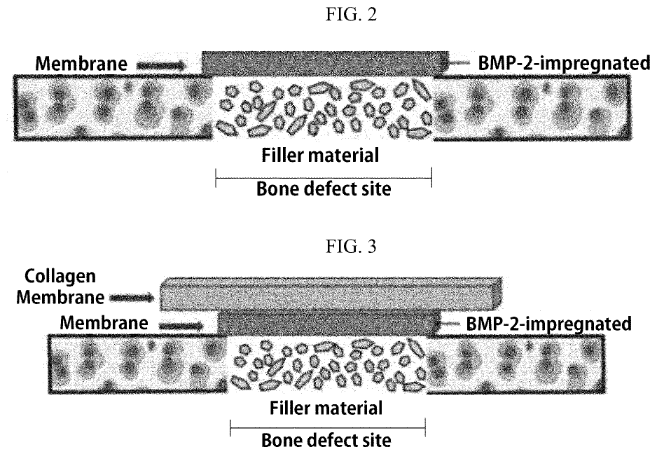

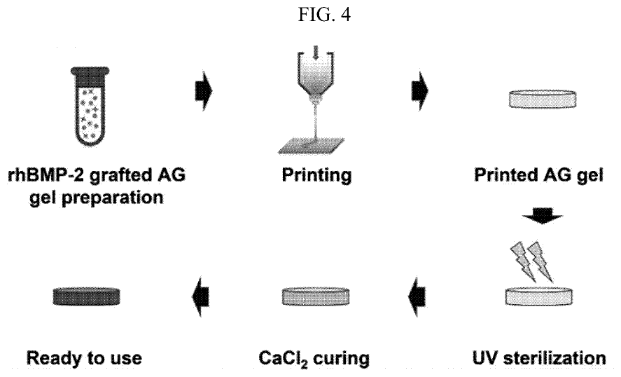

### DETAILED DESCRIPTION OF THE INVENTION

The present invention relates to a membrane for guided bone regeneration and a method for manufacturing the same.

The present invention provides a membrane for guided bone regeneration including a hydrogel containing alginate and gelatin in a weight ratio of 1:1.0-1.6, and impregnated with a bone morphogenetic protein (BMP).

Alginate is biocompatible and provides mechanical strength by crosslinking with calcium ions.

In the present invention, the term “alginate” may be used interchangeably with alginate salts such as sodium alginate, potassium alginate, or calcium alginate.

In the present invention, the term “alginate” may be used interchangeably with sodium alginate, potassium alginate, or calcium alginate.

Gelatin refers to denatured collagen and may provide anchoring of cells to integrins, thereby promoting cell-matrix adhesion, and may undergo gelation depending on temperature changes.

Alginate does not provide cell adhesion ligands, but the addition of gelatin promotes cell adhesion and differentiation.

A ratio of alginate to gelatin may affect viscosity, mechanical properties, and biological function, as well as injectability and the release kinetics of the bone morphogenetic protein (BMP).

In the present invention, the concentration of alginate may be 3 to 10% (w/v), for example, 4 to 10% (w/v), 4 to 9% (w/v), 4 to 8% (w/v), 4 to 7% (w/v), 4 to 6% (w/v), 5 to 10% (w/v), 5 to 9% (w/v), 5 to 8% (w/v), 5 to 7% (w/v), 5 to 6% (w/v), 6 to 10% (w/v), 6 to 9% (w/v), 6 to 8% (w/v), 6 to 7% (w/v), 7 to 10% (w/v), 7 to 9% (w/v), 7 to 8% (w/v), or 8 to 9% (w/v).

In the present invention, the concentration of gelatin may be 5 to 17% (w/v), for example, 5 to 17% (w/v), 5 to 16% (w/v), 6 to 17% (w/v), 6 to 16% (w/v), 7 to 17% (w/v), 7 to 16% (w/v), 8 to 17% (w/v), 8 to 16% (w/v), 9 to 17% (w/v), 9 to 16% (w/v), 9 to 15% (w/v), 10 to 17% (w/v), 10 to 16% (w/v), 10 to 15% (w/v), 11 to 17% (w/v), 11 to 16% (w/v), 11 to 15% (w/v), 12 to 17% (w/v), 12 to 16% (w/v), 12 to 15% (w/v), 12 to 14% (w/v) or 12 to 13% (w/v).

Bone morphogenetic proteins (BMPs) are growth factors involved in osteogenesis and chondrogenesis.

BMPs may be BMP-2, BMP-4, BMP-6, BMP-7, or BMP-9, and preferably BMP-2.

In one embodiment, the concentration of BMP in a mixture prepared by step (b) may be 0.1 to 1.0 mg/mL, for example, 0.1 to 0.9 mg/mL, 0.1 to 0.8 mg/mL, 0.1 to 0.7 mg/mL, 0.1 to 0.6 mg/mL, 0.1 to 0.5 mg/mL, 0.1 to 0.25 mg/mL, 0.2 to 1.0 mg/mL, 0.2 to 0.9 mg/mL, 0.2 to 0.8 mg/mL, 0.2 to 0.7 mg/mL, 0.2 to 0.6 mg/mL, 0.2 to 0.5 mg/mL, 0.25 to 1.0 mg/mL, 0.25 to 0.9 mg/mL, 0.25 to 0.8 mg/mL, 0.25 to 0.7 mg/mL, 0.25 to 0.6 mg/mL, 0.15 to 0.5 mg/mL, 0.15 to 0.4 mg/mL, or 0.15 to 0.35 mg/mL. These concentrations are lower than the 1.5 mg/mL concentration of BMP-2 approved for clinical use for therapeutic purposes.

In one embodiment, the BMP may be rhBMP-2.

The present invention provides a dual-syringe mixer including a first syringe filled with a hydrogel preparation powder including alginate and gelatin in a weight ratio of 1:1.0-1.6, and a second syringe filled with a bone morphogenetic protein (BMP) solution.

Referring to FIG. 53, a dual-syringe mixer 100 of the present invention includes a first syringe 10, a second syringe 20, and a mixing tube 30 configured to connect the two syringes so that the contents inside the two syringes may move relative to each other. The first syringe 10 contains a hydrogel preparation powder and is equipped with a first piston 11. The second syringe 20 contains a bone morphogenetic protein solution and is equipped with a second piston 21.

In one embodiment, the first syringe 10, which contains a hydrogel preparation powder, and the second syringe 20, which is filled with a bone morphogenetic protein solution, may be connected through the mixing tube 30, and the steps of pressing the piston 21 of the second syringe 20 to move the bone morphogenetic protein solution to the first syringe 10 and pressing the piston 11 of the first syringe 10 to move the contents to the second syringe 20 may be repeated, thereby mixing the hydrogel preparation powder and the bone morphogenetic protein solution. Once mixing is complete, the mixing tube 30 is removed, and a syringe containing the mixed hydrogel in either the first syringe 10 or the second syringe 20 may be selected and applied to the application site.

In regard to matters related to the composition and effects of the hydrogel in the dual-syringe mixer of the present invention, the detailed description provided above for the membrane for guided bone regeneration will be directly applied.

The present invention provides a method for manufacturing a membrane for guided bone regeneration, including the steps of: (a) preparing a dispersion by dispersing alginate and gelatin in a buffer at a weight ratio of 1:1.0-1.6; (b) preparing a mixture by mixing a bone morphogenetic protein (BMP) into the dispersion; and (c) forming the mixture using a syringe or a 3D printer.

Step (a) is a step of preparing a dispersion by dispersing alginate and gelatin in a buffer at a weight ratio of 1:1.0-1.6.

In the present invention, the buffer is any solution that adjusts pH and does not react with alginate and gelatin.

In one embodiment, the buffer may be phosphate buffered saline (PBS), 4 (2-hydroxyethyl)-1-piperazineethanesulfonic acid (HEPES) buffer, 2-[[1,3-dihydroxy-2-(hydroxymethyl) propan-2-yl]amino]ethanesulfonic acid (TES) buffer, or 3-(N-morpholino) propanesulfonic acid (MOPS) buffer.

The concentrations of alginate and gelatin within the above-described ranges allow the membrane for guided bone regeneration manufactured according to the present invention to effectively promote bone regeneration in bone defect sites.

In the present invention, step (a) may include an incubation step.

In the present invention, the dispersion refers to a mixture prior to gelation.

Step (b) is a step of preparing a mixture by mixing a bone morphogenetic protein into the dispersion prepared in step (a).

Step (c) is a step of forming the mixture prepared in step (b).

In one embodiment, step (c) may be performed by injection or 3D printing.

3D bioprinting technology may be used to manufacture customized implants and scaffolds that match the shape of bone defects. A key element of 3D bioprinting is the bioink, which should possess injectability, biocompatibility, mechanical stability, and the ability to achieve the desired resolution during the printing process. The membrane for guided bone regeneration of the present invention may be used as a bioink for 3D printing.

In one embodiment, step (c) may be performed by injection or 3D printing using a 23 G to 25 G needle.

Step (c) may include curing the mixture after forming.

Curing may be performed by treating the formed mixture with a calcium chloride (CaCl₂)) solution or by UV irradiation.

The membrane for guided bone regeneration manufactured according to the present invention contains a low concentration of a bone morphogenetic protein (BMP), thereby inducing bone formation more effectively and economically. In addition, it avoids the side effects associated with the use of conventional high-concentration BMP-2 solutions, such as hematoma, severe edema, dysphagia, and excessive bone formation.

The present invention provides a method for promoting bone formation using the above-described membrane for guided bone regeneration.

The method for promoting bone formation of the present invention includes the steps of: filling a bone defect site with a filler material; and covering an upper surface of the bone defect site with the membrane for guided bone regeneration to isolate the bone defect site from the outside.

In regard to matters related to the composition and effects of the membrane for guided bone regeneration in the method for promoting bone formation of the present invention, the detailed description provided above for the membrane for guided bone regeneration will be directly applied.

The upper surface of the bone defect site refers to the surface that comes into contact with the connective tissues surrounding the defect portion when the filler material is filled into the defect. The upper surface of the bone defect site may be flat or curved, depending on the three-dimensional shape of the defect portion.

When multiple bone defect sites are present, the direction of the upper surface of each defect portion may differ. For example, in the case of a bone defect site penetrating the bone, the upper surfaces may be present in both directions of the defect portion.

The membrane for guided bone regeneration may cover the upper surface of the bone defect site, thereby isolating the interior of the bone defect site, where bone formation induction is required, from the surrounding connective tissues.

The step of isolating the interior of the bone defect site from the outside may include: mixing a hydrogel preparation powder and a bone morphogenetic protein solution using the above-described dual-syringe mixer, and injecting a membrane composition for promoting bone formation into the bone defect site of a patient using either the first syringe or the second syringe of the dual-syringe mixer.

The membrane for guided bone regeneration is impregnated with a bone morphogenetic protein (BMP), which is released to an upper portion of the bone defect site during the healing period after bone grafting. Owing to the impregnated BMP, the protein is gradually released throughout the healing period.

By releasing the bone morphogenetic protein (BMP), which promotes osteogenesis, onto the upper surface of the bone defect site, bone formation is induced earlier on the upper portion than an inner portion of the defect portion. The bone initially formed on the upper portion supports the membrane, preventing its collapse and protecting the interior of the defect portion. This enables the filler material to form high-quality bone within the bone defect site and to achieve a high rate of bone formation.

Because only the membrane for guided bone regeneration contains bone morphogenetic proteins, while the filler material does not, a temporal difference in bone formation may be created between the upper and inner portions of the bone defect site.

The type of bone formed is not particularly limited and includes alveolar bone.

The filler material fills the bone defect site so as to contact the lower surface of the membrane for guided bone regeneration, thereby mechanically supporting the membrane and suppressing the initial release of the bone morphogenetic proteins from the membrane.

The filler material maintains the volume of the bone defect site during the bone regeneration period. It is preferable that the filler material be easy to form to the shape of the defect portion and resemble the extracellular matrix of bone.

The type of the filler material is not particularly limited and may be autologous bone, allograft bone, xenograft bone, or synthetic bone.

The filler material may be made of any one selected from the group consisting of hydroxyapatite (HA), tricalcium phosphate (TCP), biphasic calcium phosphate (BCP), bioglass, calcium sulfate, calcium carbonate, calcium phosphate, polyurethane acrylate (PUA), polyvinyl alcohol (PVA), polyethylene (PE), polypropylene (PP), polyethylene glycol (PEG), polylactide-co-glycolide (PLGA), polycaprolactone (PCL), polylactic acid (PLA), polyglycolic acid (PGA), carboxylic acid, chitosan, isoleucine, ethyl ester, gelatin, collagen, or a combination thereof, but is not limited thereto.

Biphasic calcium phosphate (BCP) is a type of bone grafting material composed of a mixture of hydroxyapatite (HA) and β-tricalcium phosphate (β-TCP). The weight ratio of HA to β-TCP in BCP may be, for example, 30:70 to 70:30, and preferably 30:70 to 40:60, but is not limited thereto.

The method for promoting bone formation of the present invention is guided bone regeneration (GBR), a dental bone regeneration technique. By applying a membrane, the method prevents the surrounding soft tissues from forming faster than the bone tissue, thereby preserving the space required for bone formation.

The method for promoting bone formation of the present invention applies rhBMP-2 to the membrane rather than directly to the bone defect site. rhBMP-2 serves to induce bone formation. However, when rhBMP-2 is applied to a bone defect site, postoperative complications such as severe edema, pain, and non-specific bone formation may occur, and the transient and rapid release of high-concentration, high-dose growth factors may be a major cause of such complications. In the method for promoting bone formation of the present invention, such problems do not occur because rhBMP-2 is applied to the membrane.

Hereinafter, the present invention will be described in detail with reference to examples.

### EXAMPLE

Various alginate-gelatin composite hydrogel (AG hydrogel) precursors with different compositions were prepared and characterized to exhibit properties suitable for forming by injection or 3D printing, and their potential as BMP-2 carriers was evaluated.

Specifically, the ratio of alginate to gelatin was first optimized based on rheological properties, injectability, 3D printability, swelling, and degradability to prepare a membrane for guided bone regeneration, and the protein release kinetics of FITC-BSA and rhBMP-2 were measured.

Second, the membrane for guided bone regeneration according to the present invention was co-cultured with bone marrow-derived mesenchymal stem cells (MSCs), and cell viability, proliferation, and osteogenic differentiation characteristics were measured.

Third, the efficacy of the membrane for guided bone regeneration according to the present invention was evaluated in a rat calvarial defect model, and micro-CT analysis, histology, and immunohistochemistry (IHC) were performed to assess the efficacy for bone regeneration.

### 1. Materials

The following materials were purchased from Sigma-Aldrich: alginate (sodium alginate, low viscosity: 4-12 cP, A1112), gelatin (type A, obtained from porcine skin, G2510), calcium chloride dihydrate (C7902), alizarin red S (A5533), cetylpyridinium chloride (CPC, C0732), paraformaldehyde (PFA, 158127), and gelatin methacryloyl (GelMA, 900496).

The following materials were purchased from ThermoFisher. phosphate-buffered saline (PBS, 1× concentrated, pH 7.4), Dulbecco's modified Eagle medium, high glucose (DMEM, 11965092), minimum essential medium alpha (α-MEM, 11900016), fetal bovine serum (A5256701), trypsin-EDTA (0.05%, Gibco, 25300054), penicillin/streptomycin/neomycin (PSN) antibiotic mixture (15640055), live/dead viability/cytotoxicity kit (L3224), fluorescein isothiocyanate (FITC)-conjugated bovine serum albumin (BSA) (A23015), and alkaline phosphatase buffer (5× concentrated, AAJ62907AE).

5-bromo-4-chloro-3-indolyl phosphate/nitroblue tetrazolium chloride (BCIP/NBT) chromogenic substrate for alkaline phosphatase (ALP) staining was purchased from Promega (S37771).

Recombinant human bone morphogenetic protein-2 (rhBMP-2) was purchased from Genoss (Korea) (GEB2-10) or PeproTech (120-02).

The enzyme-linked immunosorbent assay (ELISA) kit for BMP-2 (ab277085) and the primary antibody against CD31 (ab182981) for IHC staining were purchased from Abcam.

Gelatin, alginate, and GelMA powders for ex vivo and in vivo experiments were sterilized by irradiation with a 25 kGy electron beam (E-beam).

β-TCP pellets (<1 mm in diameter) and bovine-derived collagen membranes (approximately 0.5 mm thick) were donated by Advanced Materials Hong Kong.

The primary antibody for osteocalcin (OCN) (sc-365797) was purchased from Santa Cruz, and the horseradish peroxidase-streptavidin detection system (K4065) was purchased from Dako.

### 2. Preparation of the Hydrogel

### 2-1. Control of the Composition Ratio of Alginate and Gelatin

To identify a hydrogel with an optimal alginate-gelatin composition that balances injectability and formability, the amounts of alginate and gelatin powders were adjusted.

The alginate content was fixed at 0.3 g, and the gelatin content was increased from 0.6 g to 1.0 g in 0.1 g increments. The mixing time and the number of mixing cycles were recorded from the start of mixing until gelation occurred. Specifically, one syringe filled with alginate and gelatin powders and the other syringe filled with PBS was mixed through a mixing tube until the hydrogel became cured and could no longer be extruded, thereby identifying the optimal alginate content.

As a result, complete extrusion was not achieved when the gelatin content was 0.8 to 1.0 g. However, when the gelatin content was 0.6 g, complete fusion was confirmed, with a smooth surface and an elastic, convex shape without bubbles (FIG. 38, Table 1).

TABLE 1
Mixing Time and Mixing Cycles According to Gelatin Amount

| | | | | | Optimal |
|---|---|---|---|---|---|
| | | | Mixing | Mixing | Mixing |
| Alginate | Gelatin | PBS | Time | Cycles | Cycle |
| 0.3 g | 0.6 g | 4.1 ml | 8 min 25 s | 324 cycles | 220 cycles |
| | | | | | (5 min) |
| 0.3 g | 0.7 g | 4 ml | 4 min 2 s | 135 cycles | 100 cycles |
| | | | | | (2 min 30 s) |
| 0.3 g | 0.8 g | 3.9 ml | 3 min 19 s | 92 cycles | None |
| 0.3 g | 0.9 g | 3.8 ml | 2 min 17 s | 70 cycles | None |
| 0.3 g | 1 g | 3.7 ml | 1 min 44 s | 52 cycles | None |

Thereafter, the optimal alginate concentration was identified using the same method, based on 0.6 g gelatin as the standard. As a result, the hydrogel containing 0.4 g of alginate was confirmed to be the most suitable, as it was morphologically intact and void-free (FIG. 39, Table 2), and a hydrogel having this composition ratio was designated as 8A12 G.

TABLE 2
Mixing Time and Mixing Cycles According to Alginate Amount

| | | | | | Optimal |
|---|---|---|---|---|---|
| | | | Mixing | Mixing | Mixing |
| Alginate | Gelatin | PBS | Time | Cycles | Cycle |
| 0.3 g | 0.5 g | 4.2 ml | 14 min 19 s | 1335 cycles | 380 cycles |
| | | | | | (4 min 45 s) |
| 0.4 g | 0.6 g | 4 ml | 11 min 45 s | 746 cycles | 250 cycles |
| | | | | | (4 min 30 s) |
| 0.5 g | 0.6 g | 3.9 ml | 7 min 40 s | 508 cycles | 230 cycles |
| | | | | | (3 min 50 s) |
| 0.6 g | 0.6 g | 3.8 ml | 5 min 10 s | 422 cycles | 220 cycles |
| | | | | | (2 min 30 s) |
| 0.7 g | 0.6 g | 3.7 ml | 5 min 2 s | 357 cycles | None |
| 0.8 g | 0.6 g | 3.6 ml | 3 min 42 s | 355 cycles | None |

### 2-2. Preparation of the Hydrogel

According to Table 3, each powder was dissolved in PBS to achieve x % (w/v) alginate and y % (w/v) gelatin, stirred, and incubated overnight at 37° C. to prepare an alginate-gelatin composite hydrogel.

For example, 6A10 G (Preparative Example 1) was prepared by dissolving 0.6 g of alginate and 1.0 g of gelatin in a small amount of PBS, followed by the addition of PBS to adjust the total solution volume to 10 mL. The solution was then stirred for 5 minutes and incubated overnight at 37° C.

Preparative Examples 2 and 3, and Comparative Examples 1 and 2 were prepared using the same method, except for the masses of alginate and gelatin powders.

TABLE 3

| | Hydrogel | | |
|---|---|---|---|
| Classification | Name | Alginate x % (w/v) | Gelatin y % (w/v) |
| Preparative | 6A10G | 6 | 10 |
| Example 1 | | | |
| Preparative | 4A15G | 4 | 15 |
| Example 2 | | | |
| Preparative | 8A10G | 8 | 10 |
| Example 3 | | | |
| Comparative | 3A5G | 3 | 5 |
| example1 | | | |
| Comparative | 5A5G | 5 | 5 |
| example2 | | | |

3. Ex vivo Characteristic Analysis of the Hydrogel

### 3-1. Analysis of Mechanical Properties of the Hydrogel

### (1) Compression Test

The test was performed using an Instron tester (Super Duper Multi National Conglomerates R US) according to the International Organization for Standardization (ISO 21563) procedures. Specifically, cylindrical hydrogel specimens having a diameter of 10 mm and a length of 10 mm were placed vertically on the tester, with the upper plate of the tester in contact with the surface of the hydrogel without applying a load. Stress-strain curves were recorded using the tester, and the initial compressive modulus was calculated to evaluate the physical properties and biocompatibility of the hydrogels. The test was repeated five times for each of the 6A10 G and 8A12 G hydrogel specimens.

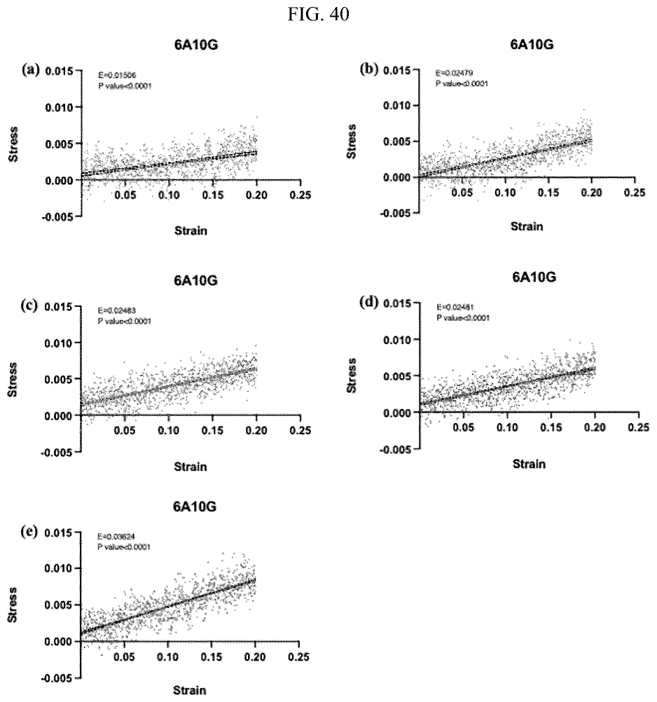

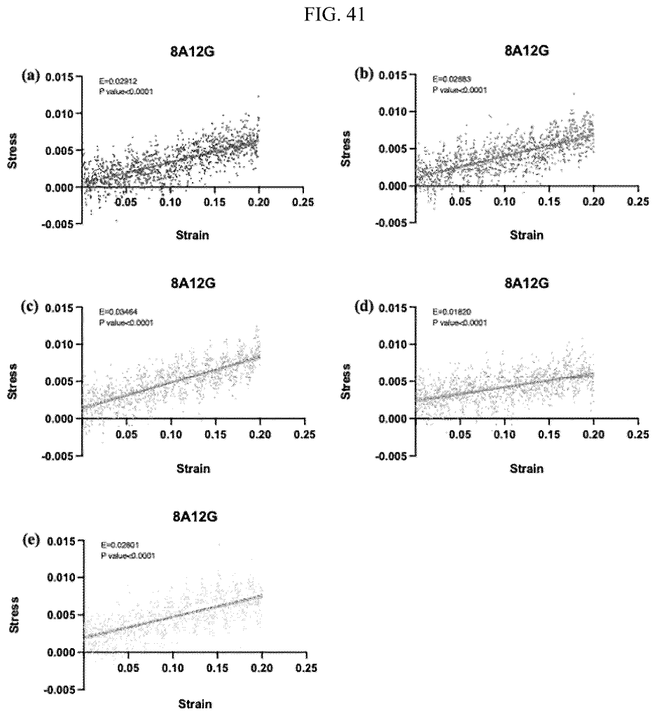

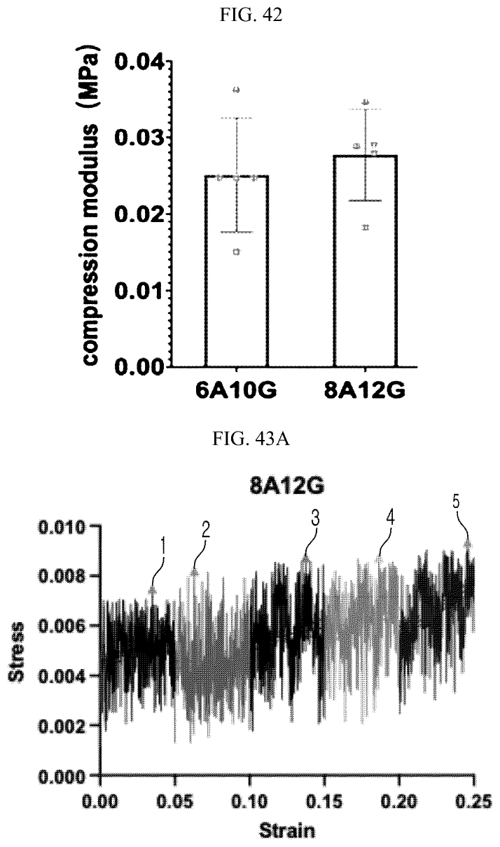

It is known that the optimal range of compressive modulus for oral hydrogels is 10 to 50 kPa (Goder Orbach et al., 2024). After the Instron test, stress-strain data with strains ranging from 0 to 2% were extracted, resulting in 1,202 data points. As a result, the compressive moduli for the 6A10 G hydrogel were 15.06, 24.79, 24.83, 24.81, and 36.24 kPa in five specimens, respectively (FIG. 40), and for the 8A12 G hydrogel, they were 29.12, 28.83, 34.64, 18.2, and 28.01 kPa (FIG. 41). These results confirmed that both 6A10 G and 8A12 G hydrogels possess compressive strength and stiffness suitable for use as membranes for guided bone regeneration (FIG. 42).

### (2) Tensile Test

The inventors analyzed the ability of the hydrogels to withstand tensile forces and deformation, and to determine their potential usability as scaffolds. Specifically, the hydrogels were poured into a standard mold according to ISO 21563 and then washed with a 0.2 M CaCl₂) crosslinking solution to induce gelation. The hydrogels were then carefully removed from the mold, and the crosslinking reaction was allowed to proceed for 20 minutes by gently shaking the dish to ensure sufficient contact between the samples and the CaCl₂) solution. After completion of crosslinking, the samples were washed twice with PBS and transferred to a new dish containing a small amount of PBS to maintain a hydrated state. The tensile testing machine (Instron, Super Duper Multi National Conglomerates R Us) was set to a crosshead speed of 1.0 mm/min. The hydrogel specimens were placed on the tensile testing machine, which was equipped with non-slip grips to ensure stable specimen holding. The specimens were aligned so that the loading direction and the fixed axis were perpendicular, and the specimen axis was centered. Each specimen was pulled until failure, and the maximum value was recorded. The tensile strength (o) was calculated according to the following ISO standard equation.

$$σ = \frac{F \times 1000}{A} ( kPa )$$

(In equation above, F denotes the force (N), and A denotes the cross-sectional area of the specimen (mm²).)

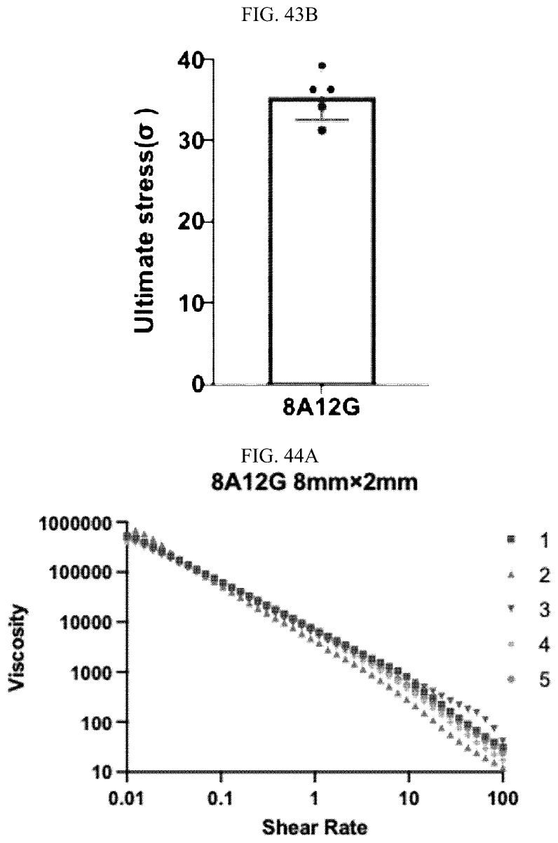

The experimental results for the five 8A12 G hydrogel specimens showed tensile strengths of 31.25, 34.17, 36.25, 36.25, and 39.17 kPa, respectively (FIGS. 43A and 43B). All of these values satisfied the ISO standard (σ≥30 kPa), confirming that the 8A12 G hydrogel has a tensile strength suitable for use in guided bone regeneration.

### (2) Viscosity Test

Viscosity is an important indicator for evaluating the injectability of the hydrogel. Measurements were performed using a standard rotational rheometer at low to high shear rates.

Specifically, 8A12 G hydrogel specimens of three different thicknesses (8 mm×1 mm, 8 mm×2 mm, and 8 mm×3 mm, n=5), prepared in the same manner as in Example 2, were tested at shear rates of 0.01-100 s⁻¹, under conditions of 25° C. (room temperature) using a PP25 measurement geometry, and a 1.0 mm gap.

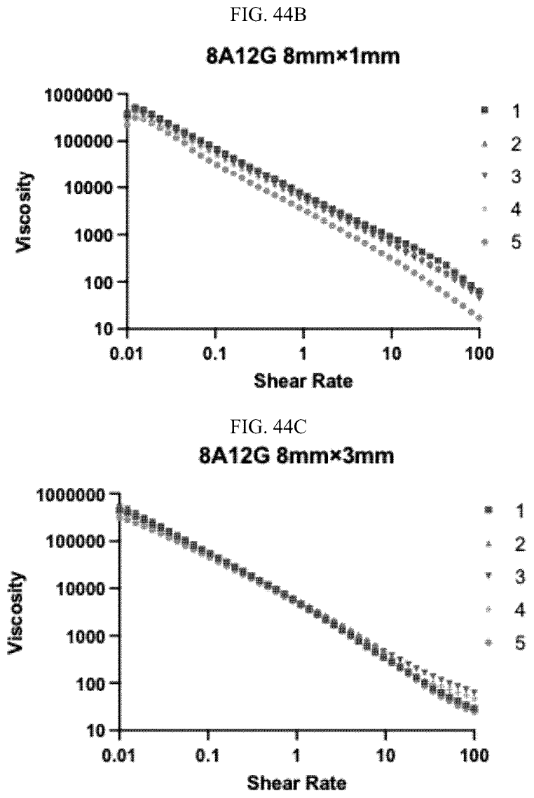

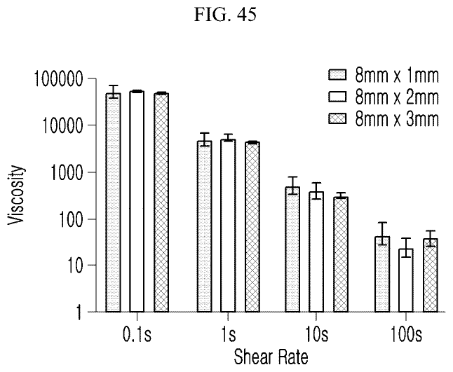

As a result, the viscosity of the hydrogel decreased markedly with increasing shear rate. The viscosity decreased from approximately 10⁶ Pa·s at low shear rates to approximately 102 Pa·s at high shear rates. Five specimens of the same thickness exhibited nearly identical viscosity profiles, with only minor differences observed, and consistent results were also obtained among the 1 mm, 2 mm, and 3 mm thick specimens (FIGS. 44A to 45). These results confirm that the 8A12 G hydrogel possesses ideal rheological characteristics for use as a membrane for guided bone regeneration, demonstrating excellent injectability, structural stability after grafting, and reproducibility.

### 3-2. Rheological Analysis of the Hydrogel

Rheological analysis was performed on the hydrogel prepared according to Example 2.

First, gelation kinetics experiments were conducted by setting the platform temperature to 25° C. to simulate temperature changes during the preparation and handling of the hydrogel for injection or 3D printing. Specifically, time sweep and shear thinning tests were performed on a rheometer (Kinexus Prime lab+, NETZSCH) equipped with 25 mm diameter plates arranged in a plate-to-plate configuration with a 0.2 mm gap. The hydrogel was uniformly placed between the upper and lower plates prior to testing.

In the time sweep test, a constant strain of 1% and a frequency of 1 Hz were applied to the hydrogel sample between the plates.

In the shear thinning tests, the hydrogel samples were sequentially sheared for 8 cycles at 1% strain (for 60 s) and at 400% or 600% strain (for 60 s), and the recovery of the storage modulus (G′) and loss modulus (G″) over time was recorded at a fixed frequency of 1 Hz. All measurements were performed in triplicate, and the average value and standard deviation were recorded.

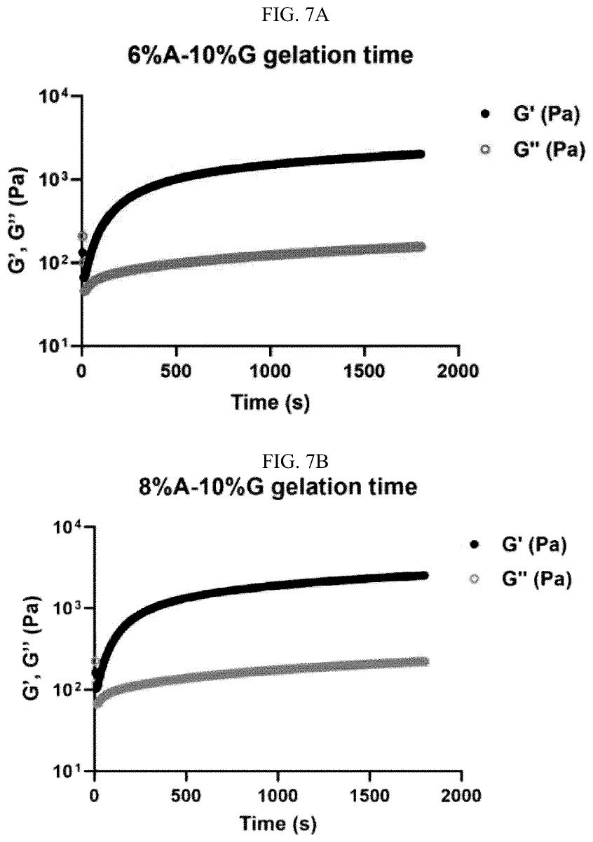

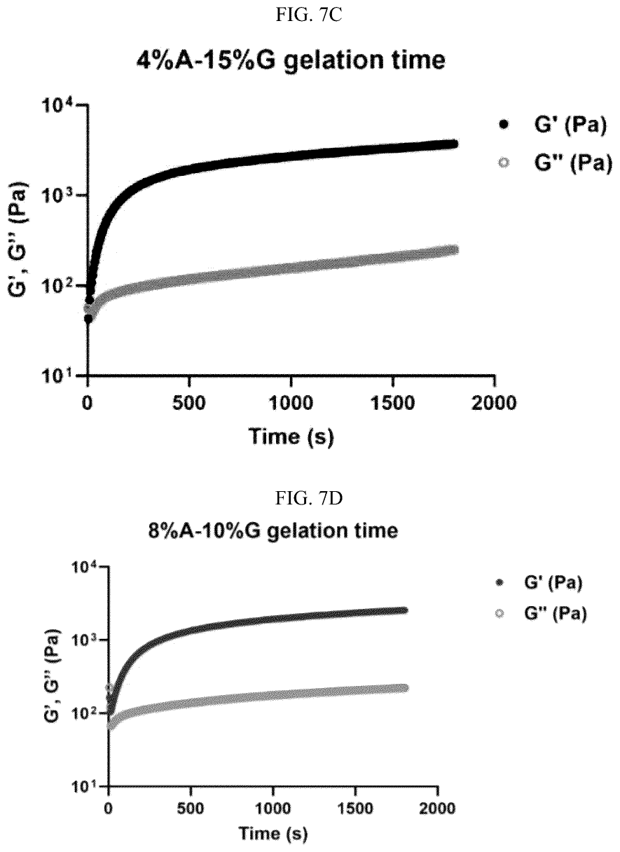

Experimental results showed that after equilibration for 1500 s on the operating platform, 3A5 G and 5A5 G exhibited relatively low storage moduli (approximately 100 Pa), indicating that they were unsuitable for tissue repair. In contrast, 6A10 G, 8A10 G, and 4A15 G exhibited significantly increased storage moduli of 1000 Pa or more (1000-3000 Pa) after 1500 s of equilibration, indicating that they were suitable for manufacturing membranes for guided bone regeneration (FIGS. 7A to 7E).

As a result of the gelation time analysis, both G′ and G″ increased over time, and a “sol-gel” transition was observed from the beginning (0 min) for 6A10 G, 4A15 G, and 8A10 G, where G′>G″=100 Pa at 25° C. (FIGS. 7A to 7E). However, 3A5 G and 5A5 G did not exhibit a “sol-gel” transition under the same conditions (FIGS. 7A to 7E).

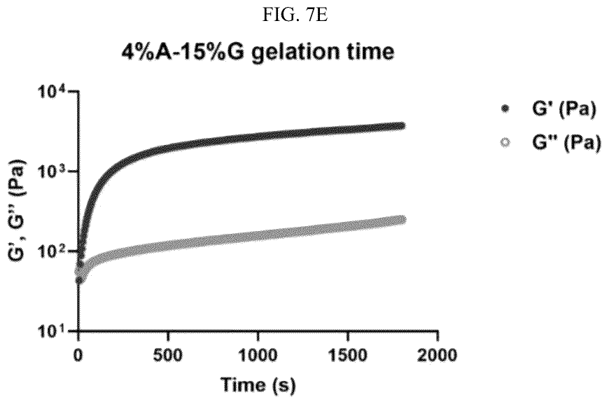

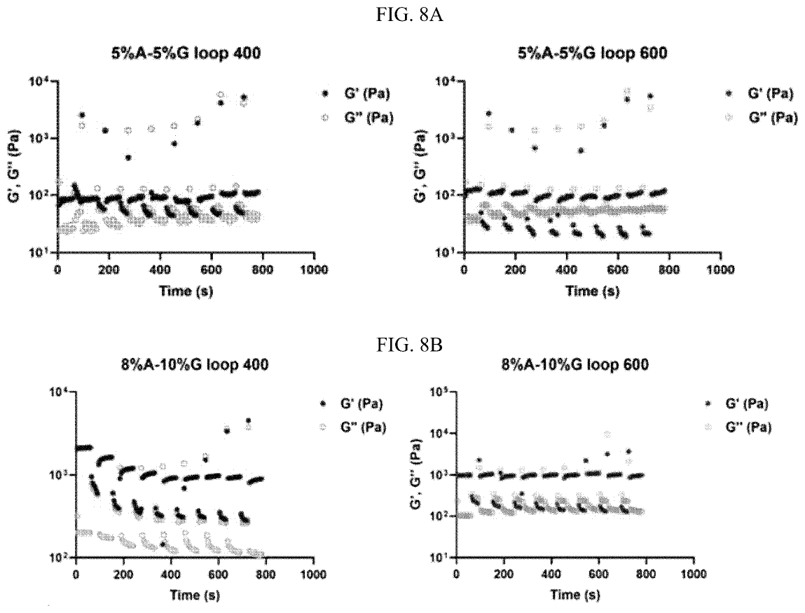

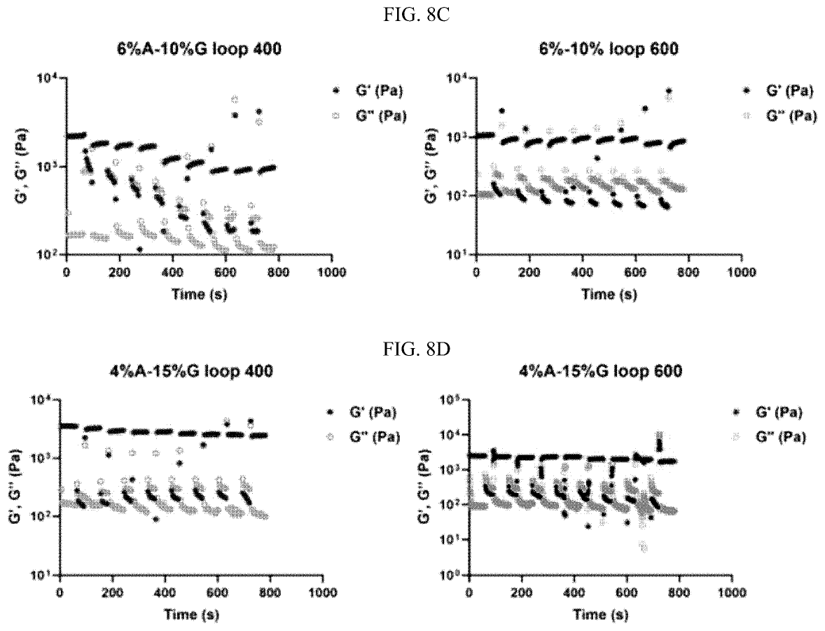

Shear peeling test results showed that all experimental groups (5A5 G, 6A10 G, 8A10 G, and 4A15 G) exhibited fluid-like behavior at high shear strains (400% and 600%), but returned to their original solid hydrogel characteristics after strain recovery. These findings suggest that the alginate-gelatin composite hydrogels have excellent injectability and are suitable for use as 3D printing materials (FIGS. 8A to 8D).

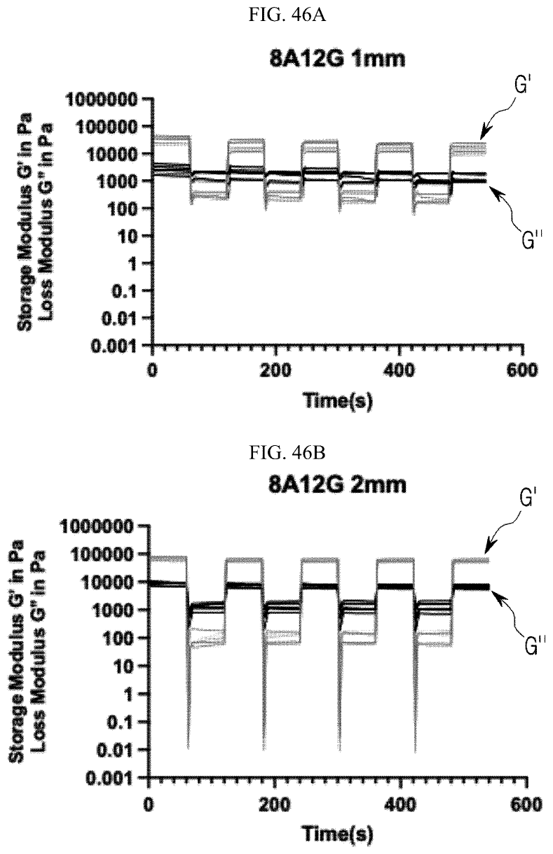

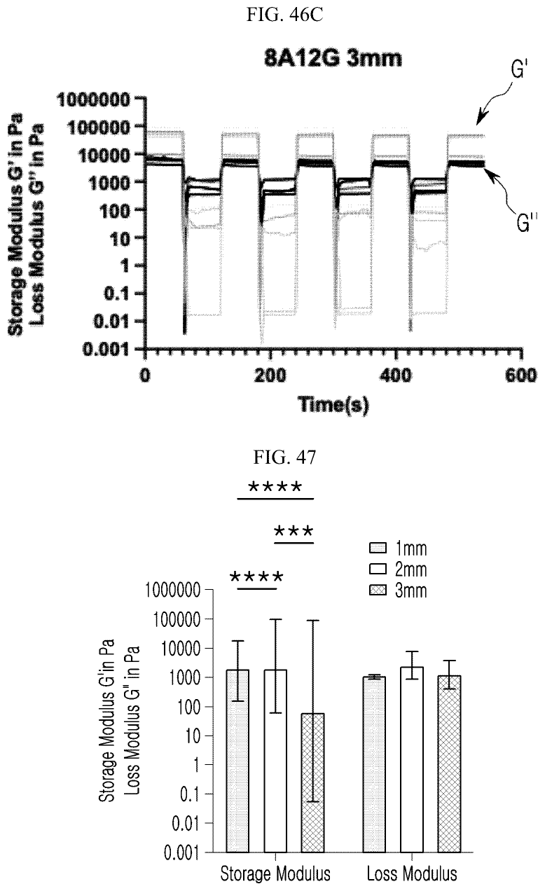

Next, to analyze the viscoelastic properties and mechanical stability of the hydrogels, five 8A12 G hydrogel specimens of three thicknesses (1 mm, 2 mm, and 3 mm) were prepared, and frequency sweep tests were performed under the following conditions: gap of 1.0 mm, measurement geometry PP08, shear strain from 1% to 400% repeated four times and returned to 1%, frequency 1 Hz, and temperature 25° C. (FIGS. 46A to 46C).

As a result, in the 1 mm-thick specimens, G′ and G″ closely overlapped and remained smooth, indicating that the 1 mm-thick hydrogel exhibits elasticity, a stable network structure, and excellent reproducibility. The 2 mm and 3 mm-thick specimens showed similar overall trends, but abrupt drops or noise were observed in some regions (FIGS. 46A to 46C). In other words, the 1 mm specimens exhibited less variability and were more stable compared to the 2 mm and 3 mm specimens. This indicates that the 8A12 G hydrogel possesses excellent mechanical strength and shape retention.

### 3-3. Injectability and Water Resistance of the Hydrogel

From the “Theological analysis of the hydrogel,” one hydrogel each was selected from those confirmed to be suitable for manufacturing membranes for guided bone regeneration (Preparative Examples 1 to 3) and those found to be unsuitable (Comparative Examples 1 and 2), and their injectability and water resistance were further evaluated.

The selected hydrogels (Preparative Example 1 and Comparative Example 2) were loaded into a 1 mL Norm-Ject syringe (Air-Tite Products, US) before gelation, and injectability was assessed by manually injecting them onto a glass slide (CITOGLAS) through a 23 G needle. The injected hydrogels on the glass slide were then immersed in distilled water or flushed with distilled water, and their appearance was examined to evaluate water resistance.

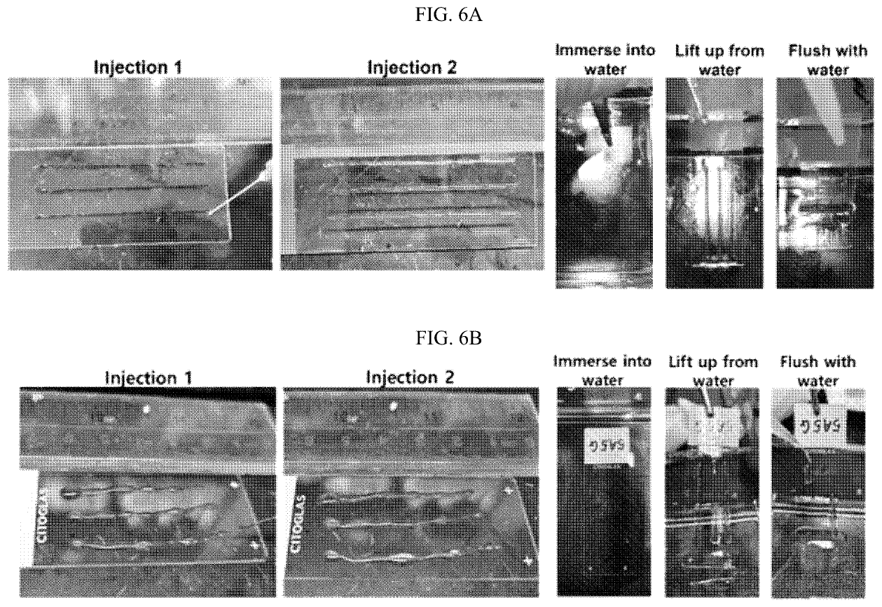

As a result, the 6A10 G hydrogel injected onto the glass slide formed uniform and consistent struts with a width of less than 1 mm, demonstrating appropriate injectability (FIG. 6A). In addition, the 6A10 G hydrogel exhibited excellent water resistance, maintaining robust stability even when immersed in or flushed with distilled water (FIG. 6A).

In contrast, the 5A5 G hydrogel showed a liquid-like behavior during injection and formed an irregular shape after gelation on the glass slide (FIG. 6B). Furthermore, its low stability in water indicated relatively poor water resistance (FIG. 6B).

### 3-4. Characteristics of the Hydrogel Depending on Curing

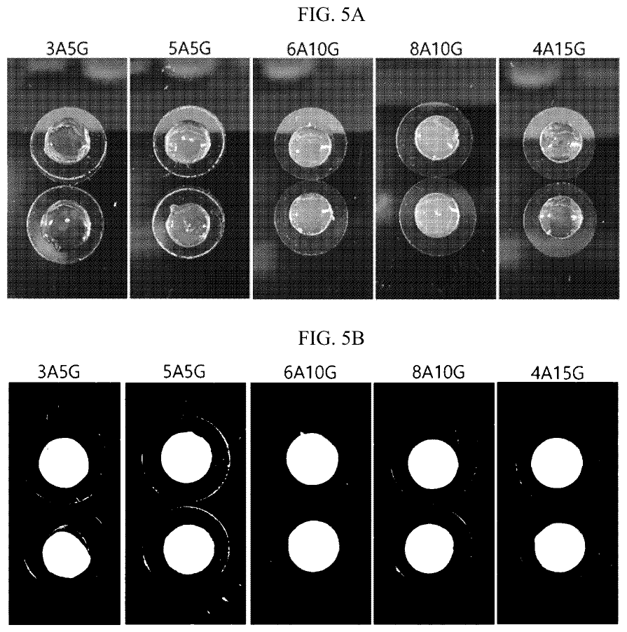

The hydrogels prepared according to Example 2 were injected into a circular mold at room temperature and observed after 10 minutes. As a result, Preparative Examples 1 to 3 exhibited a transparent and uniform disc shape, whereas Comparative Examples 1 and 2 showed irregular shapes (FIG. 5A).

To cure the hydrogels, a 2% (w/v) calcium chloride (CaCl₂)) solution was applied to the hydrogels for 15 minutes. Upon observation, the cured hydrogels turned white and opaque and were more solid compared to the uncured hydrogels (FIG. 5B).

### 3-5. Swellability of the Hydrogel Depending on Curing Time

To determine the appropriate curing time, the swellability of the hydrogel was measured according to curing time.

The volumes of 6A10 G and 4A15 G hydrogels (diameter 8 mm, thickness 1 mm, n=6) prepared according to Example 2 were measured before curing to set the baseline volume, and after curing, they were incubated in PBS under condition of 37° C. and further measured on days 0, 1, and 2. The baseline volume of the hydrogels was equal to the volume of the mold. The hydrogels were cured in 2% CaCl₂) for 15 or 30 minutes. After curing the hydrogels were washed twice with PBS, allowed to float freely in PBS, and incubated at 37° C. for 24 and 48 hours. The remaining liquid on the sample surface was then removed, and the diameter and thickness of the swollen samples were recorded. The volumetric swelling ratio was calculated as the ratio of the swollen hydrogel volume to the baseline volume.

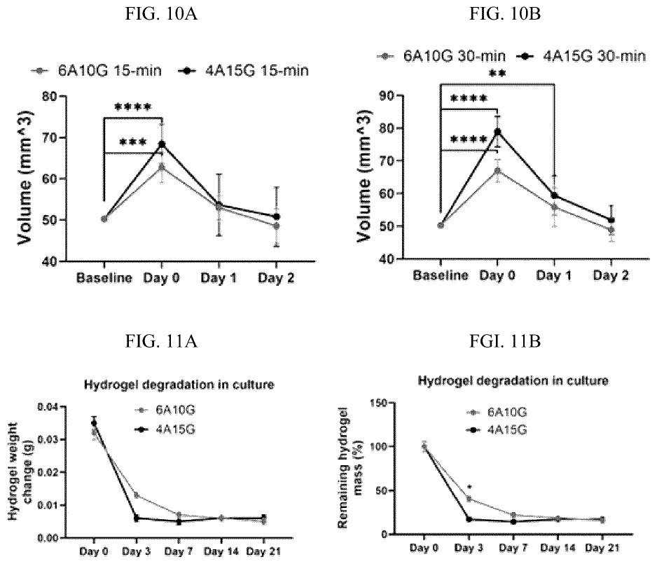

As a result, the average volumes of 6A10 G and 4A15 G hydrogels measured on day 0 after curing for 15 minutes and washing with PBS increased by 24.79% (P<0.001) and 36.15% (P<0.0001), respectively, relative to the baseline volume. However, after curing for 15 minutes, the average volumes of 6A10 G and 4A15 G hydrogels decreased by 15.56% and 21.55% on day 1, and then decreased by an additional 8.33% and 5.38% on day 2, respectively (FIGS. 10A and 10B). Ultimately, the volumes of both 6A10 G and 4A15 G hydrogels returned to their original volumes after 2 days.

In addition, the average volumes of 6A10 G and 4A15 G hydrogels measured on day 0 after curing for 30 minutes and washing with PBS increased by 33.36% (P<0.0001) and 57.04% (P<0.0001), respectively, relative to the baseline volume. Similarly, after 30 minutes of curing, the average volumes of 6A10 G and 4A15 G hydrogels decreased by 16.67% and 24.74% on day 1 compared to day 0, and by 12.43% and 12.66% on day 2 compared to day 1 (FIGS. 10A and 10B). The volumes of both hydrogels recovered to their original volumes after 2 days.

Although 15 minutes of curing resulted in slightly smaller volumes than 30 minutes of curing both conditions ultimately recovered to similar volumes after 2 days of incubation (FIGS. 10A and 10B). This similarity indicates that the two curing times have comparable effects on the swellability of the hydrogel. Based on this observation, a 15-minute curing time was preferred in subsequent processes, as it reduces processing time, minimizes the risk of contamination during hydrogel preparation, and lowers the potential for cytotoxicity arising from excessive residual calcium.

### 3-6. Characteristics of the Hydrogel Manufactured by 3D Printing

Since Preparative Examples 1 to 3 were confirmed in the previous examples to be suitable for manufacturing a membrane for guided bone regeneration, Preparative Examples 1 and 2 were selected to evaluate their applicability to 3D printing.

Hydrogels (Preparative Examples 1 and 2) were manufactured according to Example 2, and the hydrogels were printed using a hot-melt pneumatic dispenser on a Dr. INVIVO 4D2 printer (ROKIT Healthcare, Korea) by a circular extrusion method.

A cylindrical membrane having a diameter of 8 mm and a height of 0.5 mm was designed using New Creator K software (ROKIT Healthcare) and saved as a G-Code file. The G-Code was then transferred to the printer before printing. The hydrogel precursor was loaded into the dispensing barrel, and the bed temperature was set to 4° C. 4A15 G was printed at a controlled extrusion speed of 5 mm/s using a ¼-inch, 24 G nozzle at a constant pressure of 14 kPa, while maintaining the barrel temperature at 35° C. 6A10 G was extruded at a controlled extrusion speed of 5 mm/s using a ½-inch, 25 G nozzle at a constant pressure of 53 kPa, with a barrel temperature of 37° C. The printing path started with an outer circle and filled the interior with a pattern crossing the inner region. After the printing process, the hydrogel was sterilized by exposure to ultraviolet light (wavelength 365 nm) for 30 minutes. The 3D-printed hydrogel was then cured in 2% CaCl₂) for 15 minutes.

The resting time window and product yield were recorded. The resting time window refers to the time until the last printed sample undergoes gelation during 3D printing of the hydrogel precursor at room temperature. The product yield was determined as the volume of the printed hydrogel relative to the total volume of the hydrogel precursor.

The experimental results demonstrated that both 6A10 G and 4A15 G hydrogels are applicable to 3D printing and can be fabricated into hydrogels suitable for use as membranes for guided bone regeneration.

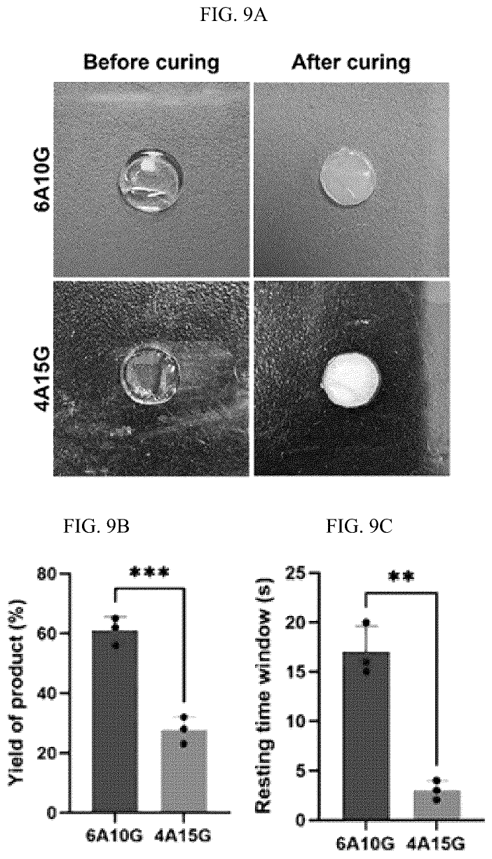

The 6A10 G hydrogel exhibited a longer resting time than the 4A15 G hydrogel (3 seconds for the 4A15 G hydrogel and 17 seconds for the 6A10 G hydrogel, FIGS. 9A to 9C).

The final product yields were 60.0% for the 6A10 G hydrogel and 27.7% for the 4A15 G hydrogel (FIGS. 9A to 9C).

### 3-7. Degradability of the Hydrogel

To evaluate the degradability of the hydrogels, 6A10 G and 4A15 G hydrogels (8 mm in diameter, 1 mm in thickness, n=6) were prepared, placed in 5 mL tubes or 24-well plates, and allowed to float freely in PBS at 37° C. They were then incubated at 37° C. for 0, 3, 7, 14, and 21 days. The hydrogels were subsequently blotted to remove any remaining liquid on the surface. The samples were lyophilized and weighed to determine their dry weight. The remaining mass ratio was calculated as the ratio of the remaining mass to the mass on day 0.

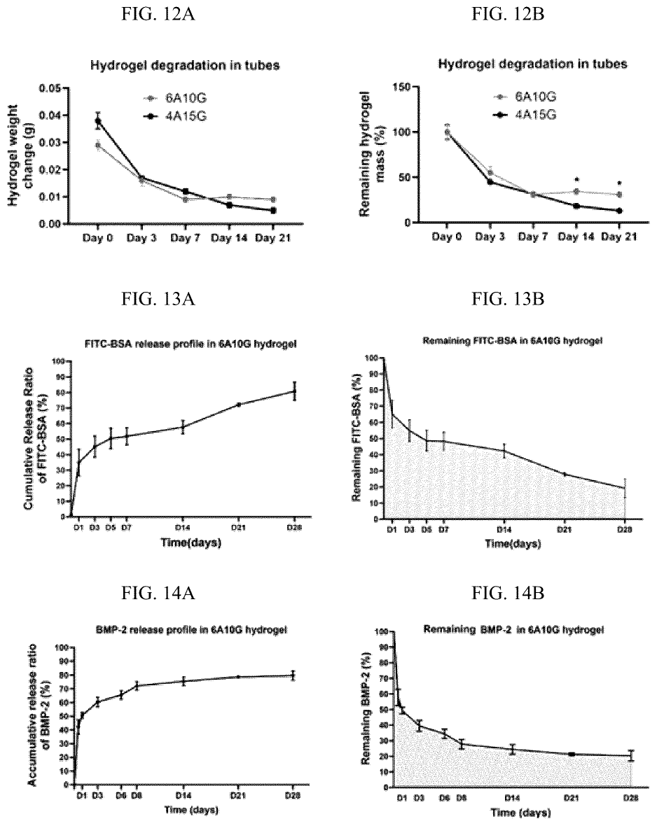

As a result, the 4A15 G hydrogel showed a faster degradation rate than the 6A10 G hydrogel in culture plates at 37° C. (FIGS. 11A to 12B). On day 3, the remaining hydrogel mass was 17.14% for 4A15 G and 40.62% for 6A10 G (P<0.05), and on day 21, it was 17.14% for 4A15 G and 15.63% for 6A10 G (FIGS. 11A and 11B).

The degradation rate of the hydrogels was lower in tubes with caps (capped tubes) maintained at 37° C. than in culture plates. In the capped tubes, the remaining hydrogel mass on day 3 was 44.74% for 4A15 G and 55.17% for 6A10 G, and on day 21, it was 13.16% for 4A15 G and 31.03% for 6A10 G (P<0.05) (FIGS. 12A and 12B).

### 3-8. Protein Release Kinetics of the Hydrogel

Fluorescein isothiocyanate-labeled bovine serum albumin (FITC-BSA) and rhBMP-2 were mixed with the 6A10 G hydrogel precursor before gelation, producing hydrogels containing 0.25 mg/mL of FITC-BSA and 0.1 mg/mL of rhBMP-2. Then, a 1 mL syringe was used to inject the hydrogel into a circular mold, producing hydrogels impregnated with a protein (FITC-BSA or rhBMP-2), each measuring 8 mm in diameter and 1 mm in thickness. To cure the hydrogel, the hydrogel was soaked in 2% CaCl₂) and incubated for 15 minutes. After rinsing twice with PBS, it was placed in 1 mL of PBS in a 1.5 mL tube. On days 1, 3, 5, 7, 14, 21, and 28, 50 μL of the supernatant was collected and replaced with 50 μL of fresh PBS.

The amount of FITC-BSA (excitation: 485 nm/emission: 518 nm) released from the hydrogel into PBS was measured over time using a fluorescence plate reader (THERMO SCIENTIFIC VARIOSKAN® Flash), and the release amount was calculated. The concentration of released FITC-BSA was determined using a linear calibration curve based on fluorescence intensity. The concentration of rhBMP-2 released into PBS was measured over time using an enzyme-linked immunosorbent assay (ELISA) kit (Abcam, Cambridge, UK) according to the manufacturer's instructions. The concentration of released rhBMP-2 was determined using a standard curve.

As a result, an initial burst release of FITC-BSA or rhBMP-2 occurred on day 1, with 34.9% or 42.1% of the total amount impregnated in the hydrogel being released (FIGS. 13A to 14B).

The release of FITC-BSA gradually slowed from day 1 to day 5 and then continued from day 7 to day 21 (FIGS. 13A and 13B). Similar to the release kinetics of FITC-BSA, the release of rhBMP-2 slowed from day 1 to day 6, after which it plateaued until day 21 (FIGS. 14A and 14B). The remaining amounts of FITC-BSA and rhBMP-2 were 19.2% and 20.4%, respectively (FIGS. 13A to 14B).

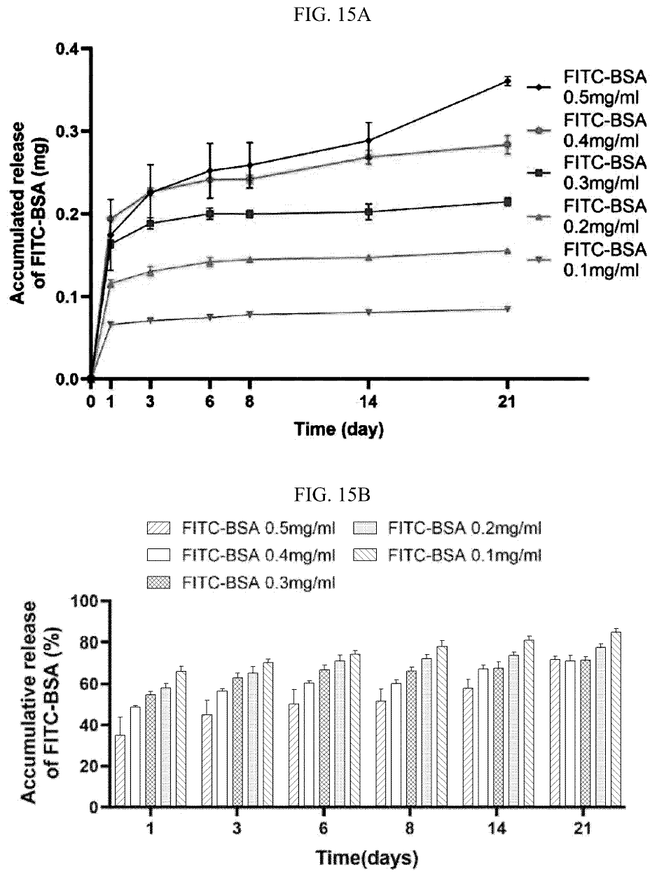

To investigate the effect of protein concentration on release kinetics, the release of FITC-BSA was evaluated at various concentrations (0.5, 0.4, 0.3, 0.2, and 0.1 mg/mL) (FIGS. 15A and 15B). The release kinetics were similar across all concentrations, exhibiting burst release (34.9%, 48.5%, 54.6%, 58.0%, and 66.0%) on day 1, followed by relatively rapid release until day 6 and then a slow, sustained release thereafter. The release of FITC-BSA was dose-dependent, with the 0.5 mg/mL FITC-BSA-impregnated hydrogel showing the highest cumulative release from day 6, and the 0.1 mg/mL hydrogel showing the lowest cumulative release (FIGS. 15A and 15B).

### 4. Cell Experiments

### 4-1. Confirmation of Hydrogel Biocompatibility

MSCs isolated from 4-week-old Sprague Dawley (SD) rats were cultured in α-minimum essential medium (α-MEM, Gibco, US) supplemented with 10% fetal bovine serum (Gibco) and 1% penicillin/streptomycin/neomycin (PSN, Gibco, US) in an incubator (37° C., 5% CO₂). The medium was changed twice a week, and MSCs were cultured until they became 80% confluent. When MSCs reached 80 to 90% confluence, the cells were detached using 1 mL of trypsin-EDTA. To assess biocompatibility, the bottoms of 96-well (80 μL) or 24-well (200 μL) plates were covered with gelled 6A10 G hydrogel or GelMA control. MSCs at a density of 1×10⁶/mL were seeded onto the surfaces of the 6A10 G hydrogel or GelMA control. After 24 or 48 h, live/dead cell staining or cell counting kit-8 (CCK-8) assays were performed in 24-well or 96-well plates, respectively, according to the manufacturer's instructions.

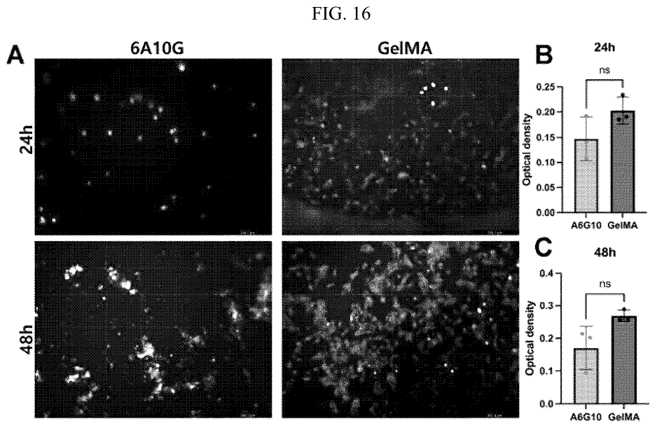

As a result, MSCs survived in both the 6A10 G hydrogel and GelMA groups, exhibiting a round morphology in the 6A10 G group and a spindle-formed morphology in the GelMA group. No significant differences were observed in cell proliferation between the 6A10 G hydrogel and GelMA group at either 24 h (P>0.05) or 48 h (P>0.05) (FIG. 16).

4-2. Confirmation of the Osteogenic Differentiation Effect of BMP-2-Impregnated Hydrogel by qPCR

MSCs were initially cultured in α-MEM-based medium (supplemented with 10% FBS and 1% PSN) in 24-well plates until confluent. The medium was then replaced with osteogenic induction medium (OIM), consisting of complete α-MEM-based medium (supplemented with 50 UM L-ascorbic acid-2-phosphate, 1 nM dexamethasone, and 10 mM β-glycerophosphate). 6A10 G hydrogels without rhBMP-2 and 6A10 G hydrogels impregnated with 0.1, 0.25, or 0.5 mg/mL rhBMP-2 were indirectly co-cultured with MSCs in a Transwell system having a pore size of 0.4 μm (Costar, US). The hydrogels were placed in the upper chamber. The cultures were maintained for 3, 7, or 21 days, with OIM replaced twice per week.

Total RNA was extracted from the samples using TRIzol reagent (Invitrogen, Carlsbad, USA). The concentration and purity of the extracted RNA were measured using a NanoDrop 2000 spectrophotometer (ND-2000; Thermo Scientific, USA). cDNA synthesis was performed using 500 ng of extracted RNA and reverse transcriptase (TaKaRa Biotechnology, Otsu, Japan) according to the manufacturer's protocol. Quantitative real-time PCR (qRT-PCR) was performed using SYBR Green Master Mix (Thermo Fisher, Waltham, USA) on an ABI 7500 sequencing detection system (Applied Biosystems, Foster City, CA, USA). Relative gene expression levels were calculated using the 2-ΔΔCt method, with GAPDH used as the endogenous control. The primer sequences used for qPCR are listed in Table 4.

TABLE 4

| | | | Amplicon |
|---|---|---|---|
| Sequence No. | Gene Name | Sequence | Length (bp) |
| (SEQ ID NO. 1) | Opn-F | TCCAAGGAGTATAAGCAGAGGGCCA | 200 |
| (SEQ ID NO. 2) | Opn-R | CTCTTAGGGTCTAGGACTAGCTTG | |
| (SEQ ID NO. 3) | GAPDH-F | AGCCCAGAACATCATCCCTG | 181 |
| (SEQ ID NO. 4) | GAPDH-R | CACCACCTTCTTGATGTCATC | |
| (SEQ ID NO. 5) | Runx2-F | GAACCAAGAAGGCACAGAC | 72 |
| (SEQ ID NO. 6) | Runx2-R | AATGCGCCCTAAATCACTG | |
| (SEQ ID NO. 7) | Col1a1-F | CAAGATGGTCGCCCTGGAC | 71 |
| (SEQ ID NO. 8) | Col1a1-R | CCAGGGAATCCCATCACACC | |
| (SEQ ID NO. 9) | Ocn-F | CTATCTGGGAGGGGTCTTCC | 71 |
| (SEQ ID NO. 10) | Ocn-R | GGTTGAGGGTGTCTGAAGGA | |

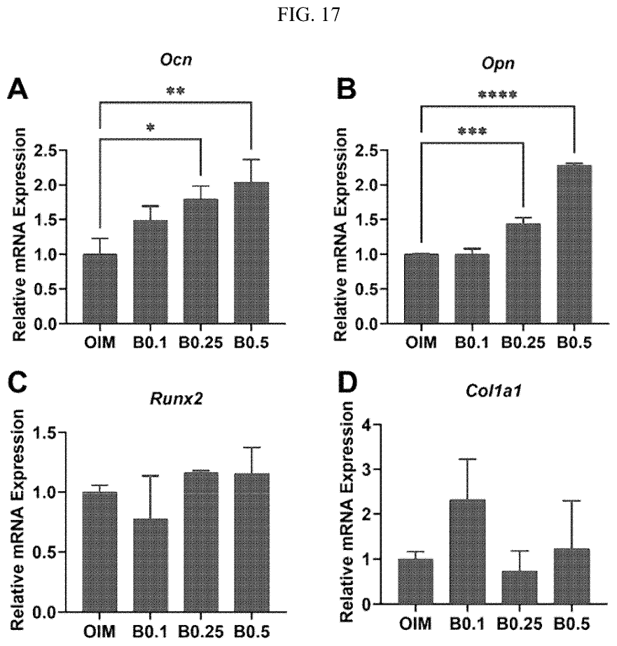

qPCR analysis showed that after 3 days of osteogenic induction, the rhBMP-2-impregnated 6A10 G hydrogels (containing rhBMP-2 at concentrations of 0.1, 0.25, and 0.5 mg/mL in hydrogel, designated as the B0.1, B0.25, and B0.5 groups) exhibited a dose-dependent upregulation of osteogenic markers, including Runx2, Ocn, and Coll, compared with the hydrogel control (FIG. 17).

### 4-3. Confirmation of the Osteogenic Differentiation Effect of BMP-2-Impregnated Hydrogel Using ALP Staining

After culturing in OIM for 7 or 21 days, cells were subjected to ALP staining. After removing the OIM, the cells were washed twice with PBS, fixed in 4% paraformaldehyde (PFA) for 15 minutes, and washed three additional times with PBS.

For ALP staining, a 5-bromo-4-chloro-3-indolyl phosphate/nitroblue tetrazolium chloride (BCIP/NBT) working solution was added to the live cells and incubated at room temperature for 20 minutes. The staining solution was then removed, and the cells were washed twice with distilled water.

After drying ALP staining images were captured using a microscope (Leica) and a scanner (Flexcell). To quantify the stained ALP, 10% (w/v) cetylpyridinium chloride (CPC) was added to lyse the stained cells, and the absorbance of the resulting solution was measured at 562 nm using a microplate reader (Molecular Devices VersaMax, Workingham) (n=6).

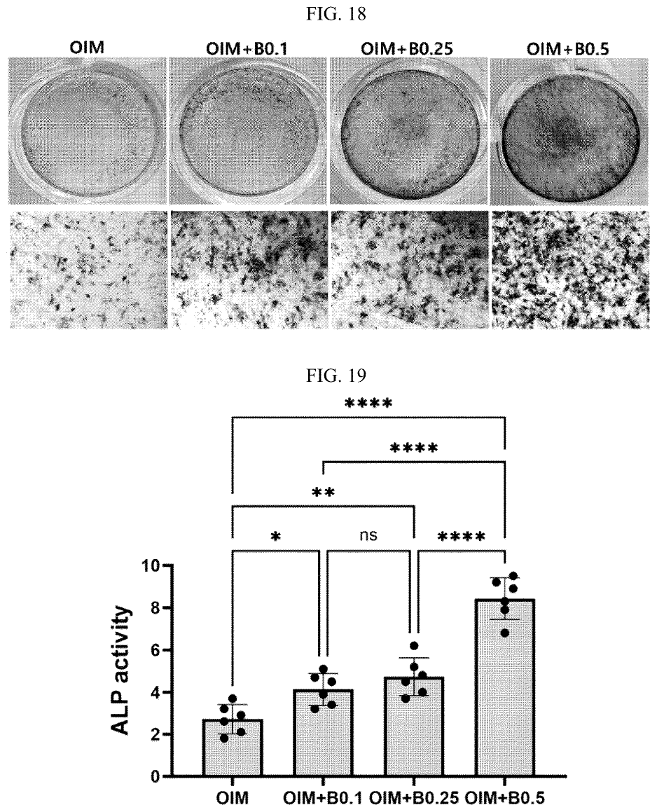

ALP staining demonstrated that rhBMP-2-impregnated 6A10 G hydrogels promoted ALP production in a dose-dependent manner. Specifically, after 7 days of osteogenic induction, ALP production increased by 52.10% (P<0.05), 74.20% (P<0.01), and 210.4% (P<0.001) in the B0.1, B0.25, and B0.5 groups, respectively, compared with the control (FIGS. 18 and 19).

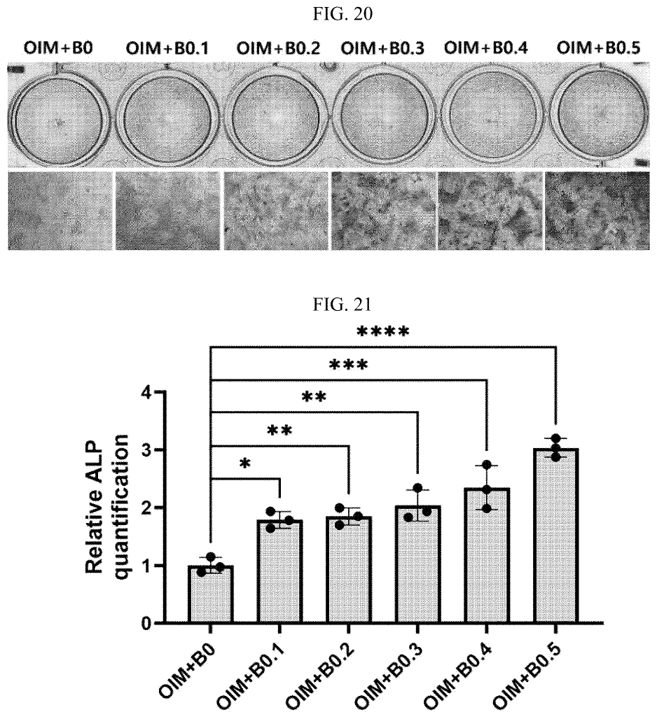

Furthermore, to validate the effect of rhBMP-2 concentration, the osteoinductive potential of rhBMP-2 at various concentrations (0, 0.1, 0.2, 0.3, 0.4, and 0.5 mg/mL) was assessed under culture conditions. After 7 days, ALP production was promoted in a dose-dependent manner by 78.0% (P<0.05), 84.1% (P<0.01), 102.7% (P<0.01), 133.6% (P<0.001), and 202.4% (P<0.0001) in the B0.1, B0.2, B0.3, B0.4, and B0.5 groups, respectively, compared with the hydrogel control (OIM+B0) (FIGS. 20 and 21).

### 44. Confirmation of the Osteogenic Differentiation Effect of BMP-2-Impregnated Hydrogel Through Alizarin Red Staining

After culturing cells in OIM for 7 or 21 days, Alizarin Red staining (ARS) was performed. After removing the OIM, the cells were washed twice with PBS and fixed in 4% paraformaldehyde (PFA) for 15 minutes. A 0.2% ARS solution (pH 4.1) was then added, and the cells were incubated at room temperature for 20 minutes. Then, the staining solution was removed, followed by washes with distilled water three times. After drying, ARS images were captured using a microscope (Leica) and a scanner (Flexcell). To quantify ARS, cetylpyridinium chloride (CPC) was added to lyse the stained cells, and the absorbance of the resulting solution was measured at 570 nm using a plate reader (n=6).

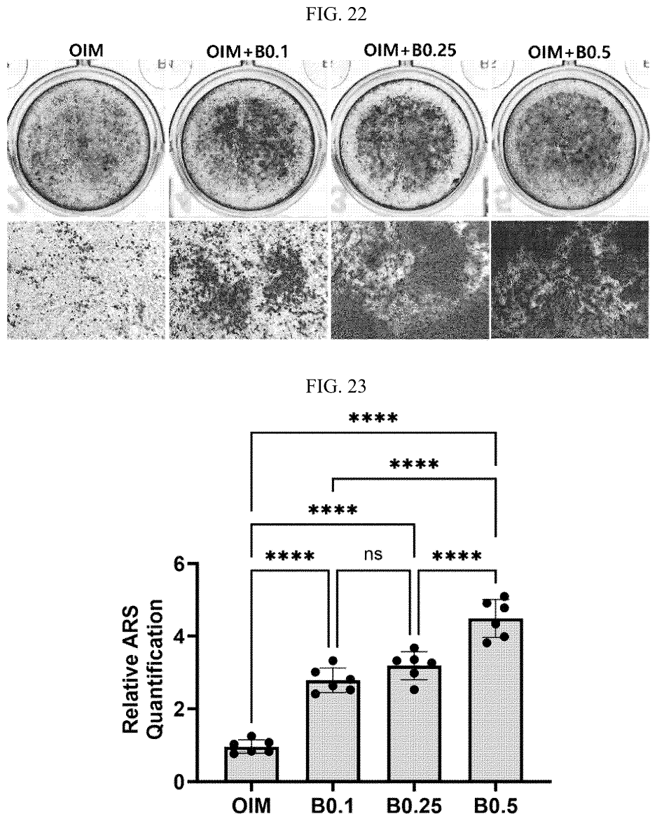

ARS staining demonstrated that rhBMP-2-impregnated 6A10 G hydrogels promoted calcium nodule formation in a concentration-dependent manner. Specifically, after 21 days of osteogenic induction, calcium nodule formation increased by 190.90% (P<0.001), 232.92% (P<0.001), and 368.80% (P<0.001) in the B0.1, B0.25, and B0.5 groups, respectively, compared with the hydrogel control (FIGS. 22 and 23).

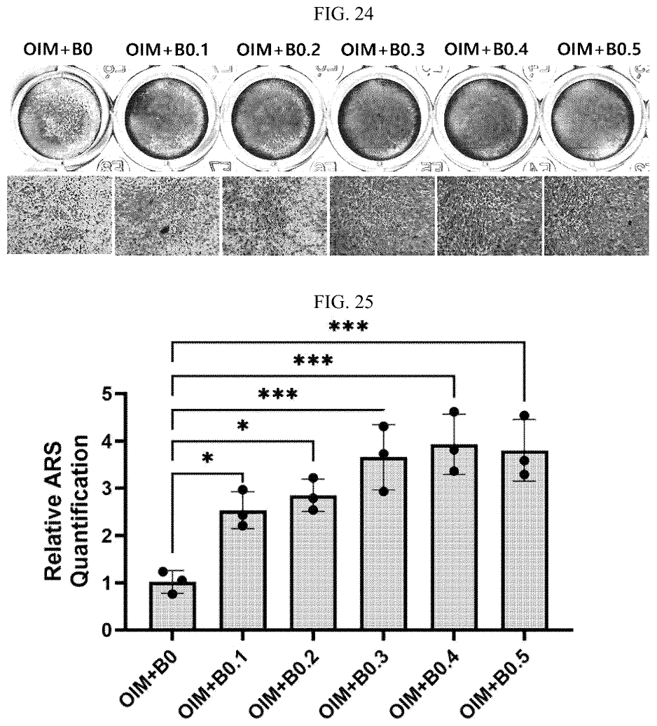

Furthermore, to verify the effect of rhBMP-2 concentration, the osteoinductive potential of mhBMP-2 at various concentrations (0, 0.1, 0.2, 0.3, 0.4, and 0.5 mg/mL) was evaluated. After 21 days, calcium nodule formation was promoted in a dose-dependent manner by 149.5% (P<0.05), 180.3% (P<0.05), 259.7% (P<0.05), 286.7% (P<0.001), and 274.1% (P<0.001) in the B0.1, B0.2, B0.3, B0.4, and B0.5 groups, respectively, compared with the hydrogel control (OIM+B0) (FIGS. 24 and 25).

5. Animal Experiment: Confirmation of the Efficacy of the Membrane for guided bone regeneration

### (1) Rat Model Experiment

Male Sprague-Dawley rats (10 weeks old) were used in this experiment. The animal experiment protocol was approved by the Animal Experiment Ethics Committee (AEEC) of the Chinese University of Hong Kong (Approval No. 19-031-HMF). All procedures were conducted in accordance with the guidelines of the National Institutes of Health (NIH). The surgery was performed under anesthesia following the procedure shown in FIG. 26. Briefly, an incision was made along the midsagittal line of the rat skull. The periosteum was carefully incised along the sagittal suture to expose the calvarial bone. While irrigating with saline, a circular defect (8 mm in diameter) was created through the midsagittal suture using a trephine drill (Dentium, Suwon, Korea). β-TCP pellets (2 mg, diameter <1 mm), donated by Advanced Materials Hong Kong, were evenly placed within the defect site as filler material.

A membrane for guided bone regeneration, specifically, a cured 6A10 G hydrogel membrane impregnated with 2.5 μg of rhBMP-2 (corresponding to a 6A10 G hydrogel membrane impregnated with 0.1 mg/mL of rhBMP-2; diameter 8 mm, thickness 0.5 mm), was placed on the surface of the filler material and designated as the guided bone regeneration experimental group (Exp GBR).

Rats that received the filler material without a membrane for guided bone regeneration were designated as the guided bone regeneration control (Con GBR).

Before final suturing, a 10 mm long, 0.5 mm thick square collagen membrane (bovine origin, donated by Advanced Materials Hong Kong) was placed over the membrane or β-TCP pellets to serve as a supporting scaffold.

The remaining rats that received no treatment were used as the blank control (BLK).

The rats were euthanized 3 or 6 weeks after surgery. The sample size for this animal study was five rats per time point

### (2) Rabbit Cranial Defect Model Experiment

Fifteen adult male New Zealand White rabbits were used in this experiment. The rabbits were individually housed in cages, provided with specialized feed and resting areas, and maintained under conditions that allowed positive olfactory and tactile interaction. Surgery was performed after shaving the cranial region and disinfecting it with povidone-iodine. Specifically, an incision was made along the midline of the skull, followed by careful separation of the periosteum and soft tissue. A circular defect portion measuring 8 mm in diameter was created using a low-speed trephine bur to simulate bone loss, with continuous saline irrigation throughout the procedure. After the defect site was thoroughly cleaned with saline and gauze, the scaffold was implanted, and the defect was subsequently covered with periosteum and soft tissue and closed with absorbable sutures. Three interrupted sutures-superior, middle, and inferior-were applied for stabilization, followed by complete skin closure using continuous sutures. The implanted scaffolds were divided into five groups: the control group (C), in which the scaffold was injected with 0.1 mL of 6A10 G hydrogel; the immediate BMP-2 group (iB), in which the scaffold was injected with 0.1 mL of 6A10 G hydrogel followed by drip-injection of BMP-2 solution (0.1 mL, 0.3 mg/mL); the BMP-2 pre-soaked scaffold group (SB), in which the scaffold was injected with 0.1 mL of 6A10 G hydrogel and then pre-soaked in 0.2 mL of BMP-2 solution (0.3 mg/mL) prior to implantation; the PDRN pre-soaked scaffold group (SP), in which the scaffold was injected with 0.1 mL of 6A10 G hydrogel and then pre-soaked in 0.2 mL of PDRN solution (0.3 mg/mL) prior to implantation; and the BMP-2-loaded hydrogel group (AB), in which BMP-2 (0.3 mg/mL) was directly incorporated into PBS during the manufacturing process of the 6A10 G hydrogel to produce 0.1 mL of BMP-2-loaded hydrogel for implantation. The rabbits were euthanized at 2, 4, or 6 weeks after surgery using Zoletil and calcium chloride.

### 5-1. Micro-CT Analysis

### (1) Rat Model Experiment

All skull specimens were fixed in 4% PFA for 48 hours and subsequently transferred to 70% ethanol. For bone mass analysis, a micro-computed tomography (viva CT, Scanco) was used with a supply voltage of 70 kV and a current of 114 μA. A total scanning depth of 5 mm with a spatial resolution of 10 μm was applied. To remove background signal noise, a low-pass Gaussian filter with a sigma of 2.5 and a support of 2 was used. Mineralized tissue was defined using a threshold of 85 Hounsfield units. The region of interest (ROI) was set as the 8-mm calvarial defect site created during surgery (n=5), centered on the sagittal suture. A volume of interest (VOI) of approximately π×4× 4 mm was generated from 25 slices, and the bone volume/total volume ratio (BV/TV) was calculated using CTAn. The reconstructed 3D images were analyzed with MicroView 3D Image Viewer (Version 2.5.0; Parallax Innovations, Canada).

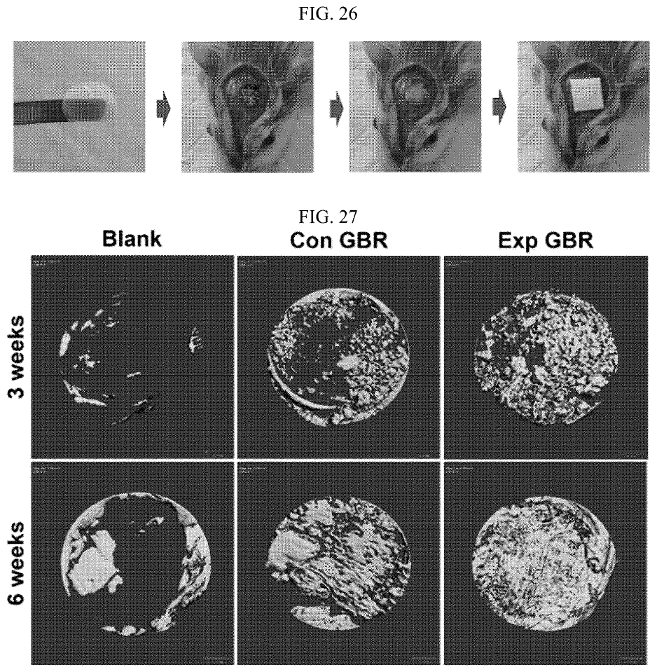

The 3D reconstructed images showed a small amount of bone mass in the blank control, a greater amount of bone mass in the GBR control than in the blank control, and the greatest amount of bone mass in the GBR experimental group (FIG. 27).

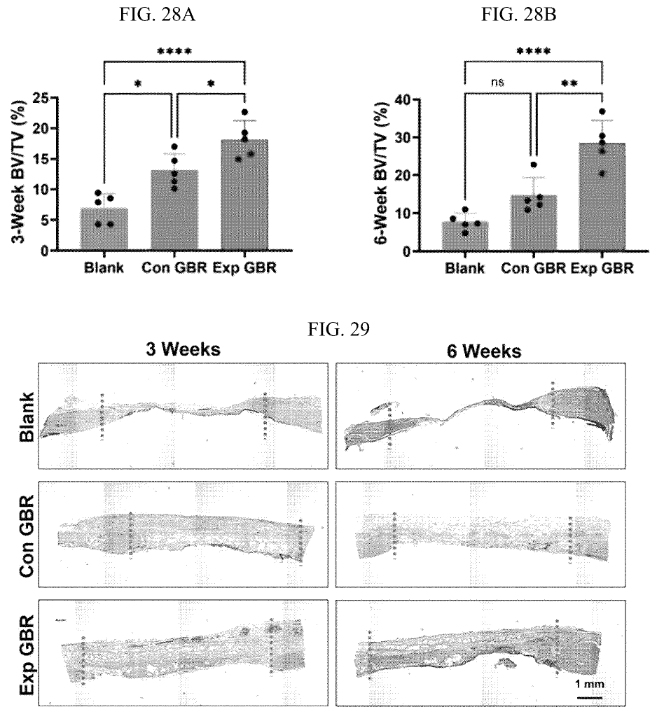

At 3 or 6 weeks after grafting the regenerated bone mass within the defect site was greater in the GBR control than in the blank control (+90.2% in BV/TV, P<0.05; +136.9% in BV/TV, P>0.05). In the GBR experimental group, the regenerated bone mass was significantly greater than that in the blank control (+163.6% in BV/TV, P<0.0001; +268.2% in BV/TV, P<0.0001) and also significantly greater than that in the GBR control (+38.6% in BV/TV, P<0.05; +94.2% in BV/TV, P<0.001) at both 3 and 6 weeks after grafting (FIGS. 28A and 28B). Newly formed bone was further confirmed through histological analysis.

### (2) Experiment Using a Rabbit Calvarial Defect Model

The defect site was marked with a drill and completely resected. The resected specimen was immediately fixed in a pre-prepared container containing 10% formalin. It was then scanned using micro-computed tomography and stored in DICOM format. The CT images were analyzed using dedicated analysis software, and 3D structural reconstructions were generated. Bone regeneration was quantitatively evaluated by calculating bone volume and percentage from the 3D reconstructions. Data were compared using the Kruskal-Wallis test.

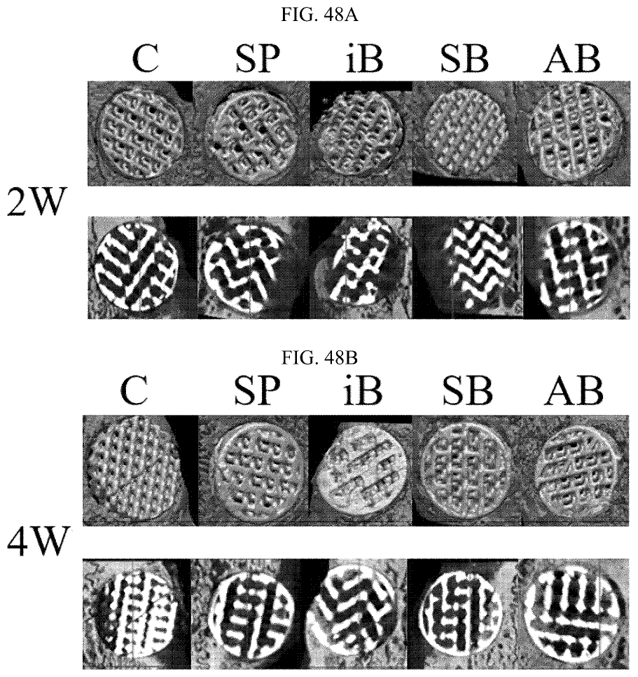

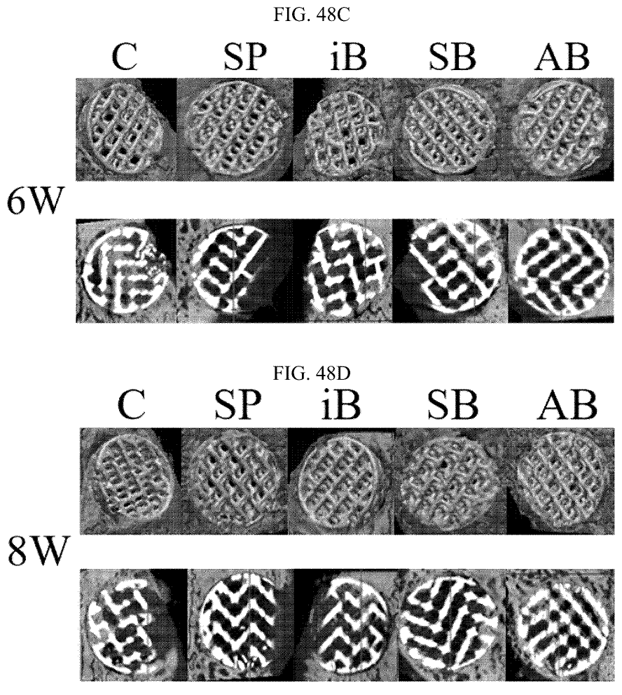

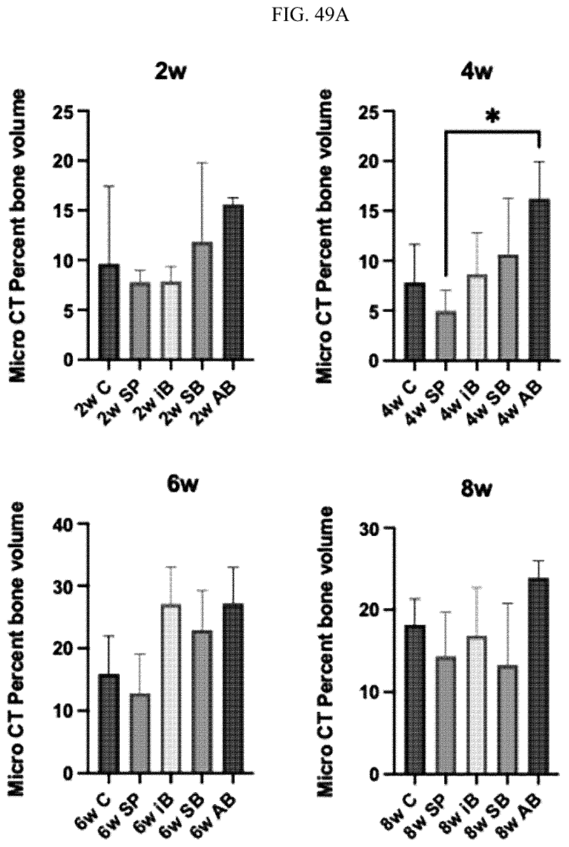

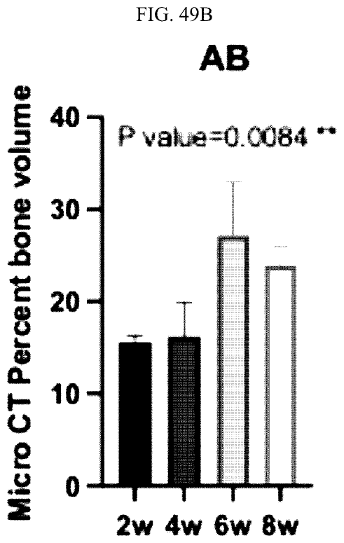

As a result, the AB group exhibited the most pronounced bone formation, demonstrating the highest bone regeneration efficacy (FIGS. 48A to 48D). The SP group showed the lowest bone regeneration, while the AB group consistently demonstrated the highest level of bone regeneration at 2, 4, 6, and 8 weeks after grafting (FIGS. 49A and 49B).

### 5-2. Histological Analysis

### (1) Rat Model Experiment

All skulls underwent histological analysis following micro-CT scanning. Skull samples were decalcified in a 10% ethylenediamine-tetraacetic acid (EDTA) solution for 4 weeks. After decalcification, the samples were dehydrated and paraffin-embedded. Five-micron-thick tissue sections were prepared along the sagittal suture using a Leica microtome. These sections were then stained with hematoxylin and eosin (H&E, Sigma-Aldrich) and examined under a Leica light microscope. The percentages of newly regenerated bone, β-TCP pellets, and fibrous tissue within the calvarial defect site (n=5 per time point) were quantified using ImageJ software (NIH, MD).

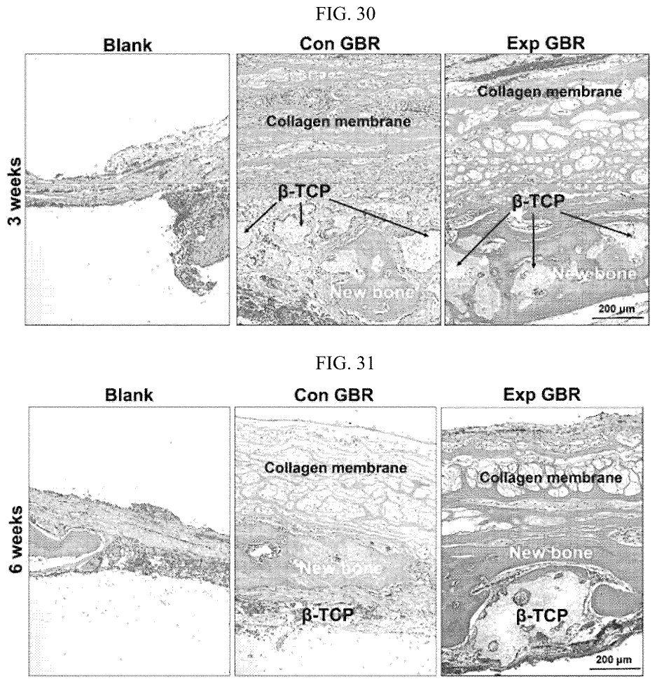

Histological analysis of the H&E-stained sections revealed that at 3 or 6 weeks after grafting, the defect site in the blank control was largely covered with fibrous tissue, and only minimal bone formation was observed at the defect margin (FIGS. 29 to 31). In both the GBR control and GBR experimental groups, the tissue within the defect site appeared substantially thicker than in the blank control. Notably, in the GBR experimental group, newly formed bone extended from the upper edge of the defect to the region beneath the collagen membrane (FIGS. 29 to 31).

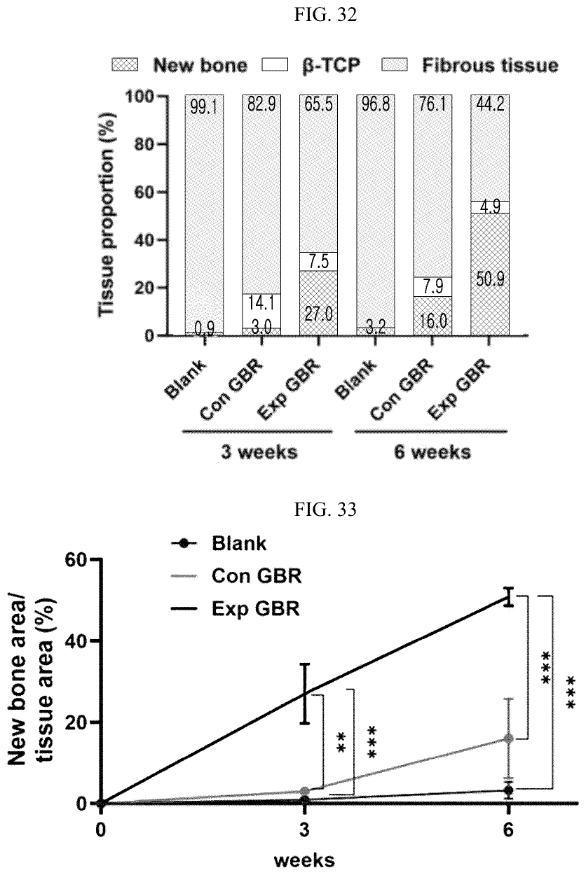

At both 3 and 6 weeks after grafting, the GBR experimental group exhibited significantly greater new bone formation than the GBR control. At 3 weeks, the proportion of newly formed bone was 27.0% in the GBR experimental group compared with 3.0% in the GBR control (P<0.01). At 6 weeks, the new bone ratio increased to 50.9% in the GBR experimental group, whereas the GBR control showed 16.0% (P<0.001) (FIGS. 32 and 33). In addition, the collagen membrane and β-TCP pellets remained within the defect site even at 6 weeks after grafting. The percentage of β-TCP pellets was relatively lower in the GBR experimental group than in the GBR control (7.48% vs. 14.11% at 3 weeks; 4.86% vs. 7.85% at 6 weeks) (FIGS. 32 and 33), indicating that pellet degradation occurred more rapidly in the GBR experimental group over time.

### (2) Experiment Using a Rabbit Calvarial Defect Model

Tissue sections were prepared using a microtome. Specimens preserved in 10% formalin were decalcified using 10% EDTA solution, 5% formic acid solution, and 3% hydrochloric acid. After decalcification, the samples were dehydrated through a graded ethanol series (80%→94%→100%), cleared with xylene, and subsequently paraffin-embedded. The paraffin-embedded specimens were then sectioned, mounted on slides, and stained with H&E. Bone regeneration and tissue responses were evaluated through microscopic observation and histopathological assessment.

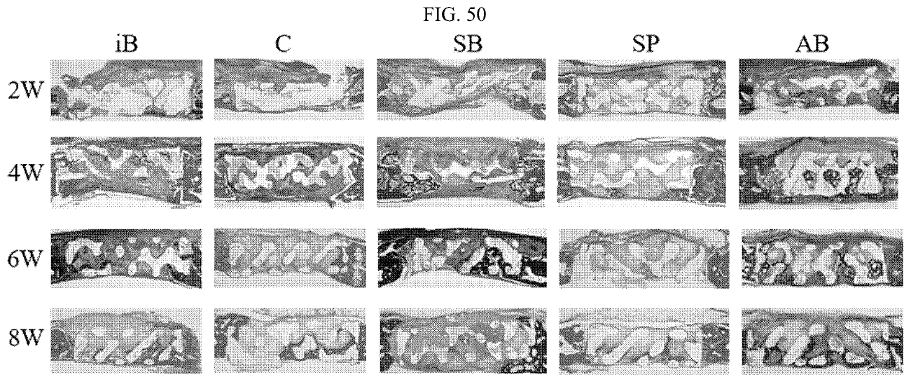

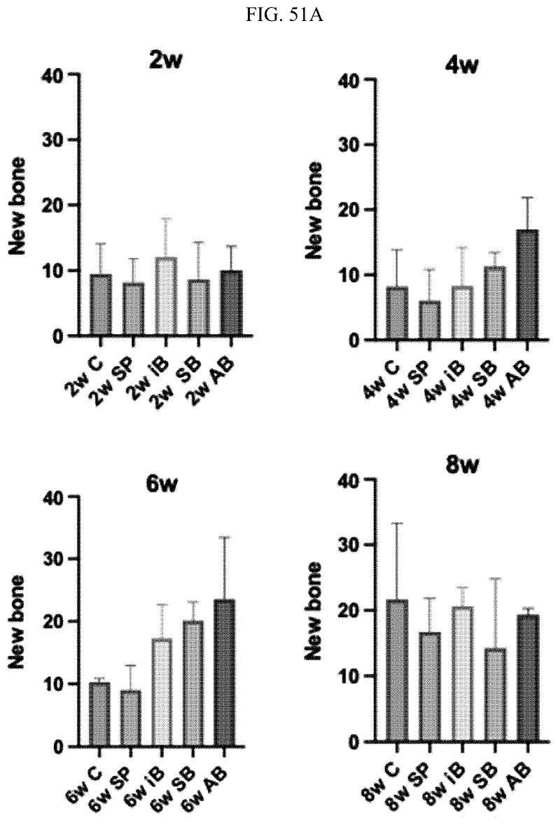

As a result, significant bone regeneration was observed between the calvarial defect and the scaffold in tissue sections from the AB group at all time points. This finding confirms that in the AB group, BMP-2 was incorporated during the preparation of the 6A10 G hydrogel, allowing the hydrogel to function as an effective carrier for BMP-2 and thereby maximizing bone regeneration (FIG. 50).

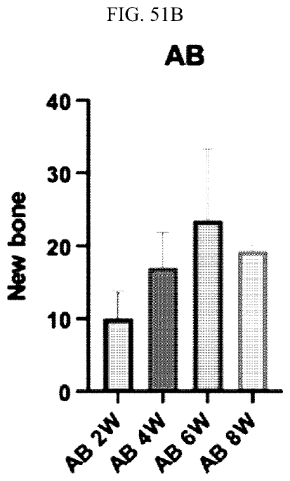

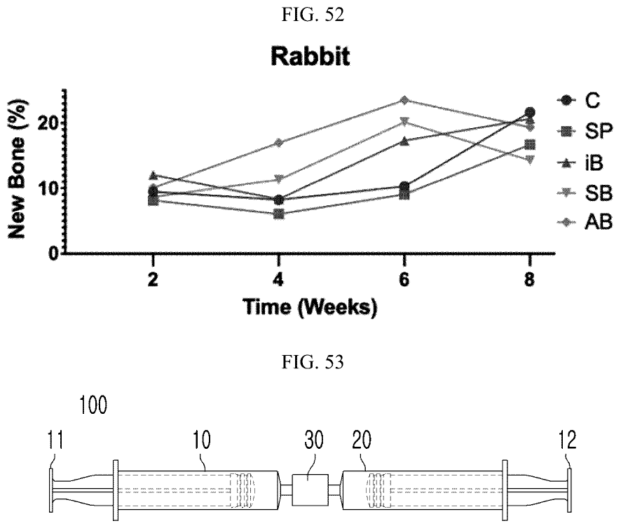

Furthermore, the Kruskal-Wallis test showed a continuous increase in bone regeneration from 2 to 6 weeks. This indicates that the presence of BMP-2 within the hydrogel effectively promotes early osteogenesis, and the enhanced bone regeneration observed during the early and mid stages suggests that BMP-2 plays a pivotal role in accelerating bone formation immediately following grafting (FIGS. 51A and 51B). Moreover, incorporating BMP-2 directly into the hydrogel during scaffold preparation enabled sustained local delivery, contributing not only to enhanced bone regeneration but also to improved structural stability within the defect site.

### 5-3. Immunohistochemistry

Tissue sections prepared in Example 5-2 were incubated overnight at 4° C. with primary antibodies.

The primary antibodies used were as follows: anti-CD31 antibody (1:200 dilution; Abcam) for endothelial cell detection and angiogenesis assessment, and anti-osteocalcin (OCN) antibody (1:100 dilution; Santa Cruz) for the identification of mature osteoblasts and evaluation of bone formation.

Positive signals were developed using a horseradish peroxidase-streptavidin detection system (Dako) following incubation with the secondary antibody. Five regions on each slide (n=5 per time point) were randomly selected under a light microscope (Leica, Germany). Image analysis was performed using ImageJ software (NIH). The region of interest (ROI) was defined as the entire tissue area within the defect site beneath the collagen membrane. Blood vessel density (CD31 positivity) was calculated by dividing the number of vessels by the total ROI area. The percentage of OCN-positive region was determined by dividing the OCN-positive region by the total ROI region.

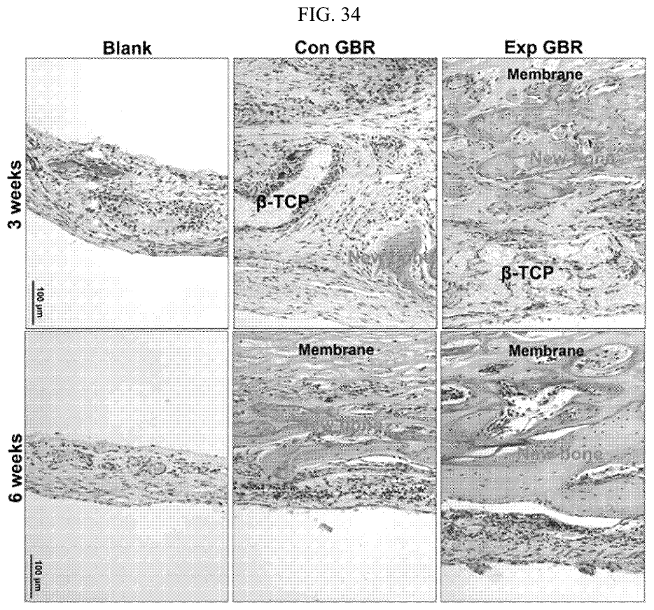

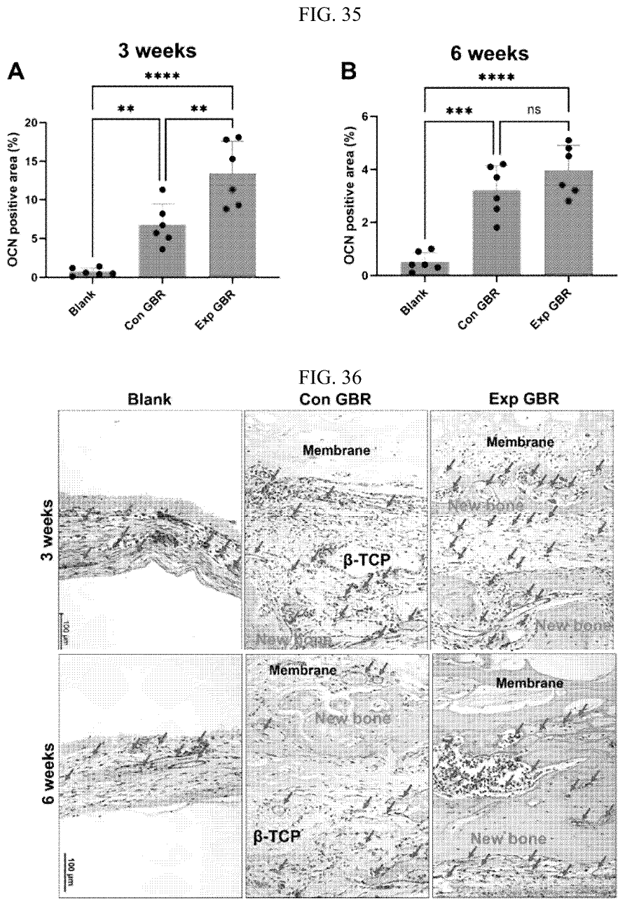

As a result, 3 weeks after grafting, OCN expression was significantly higher in both the GBR control and GBR experimental groups compared to the blank control (GBR control: +8.67-fold, P<0.01; and GBR experimental group: +18.19-fold, P<0.0001) (FIGS. 34 and 35). In addition, OCN expression in the GBR experimental group was significantly higher than in the GBR control (+98.5%, P<0.01) (FIGS. 34 and 35). At 6 weeks, both the GBR control and GBR experimental groups continued to show significantly elevated OCN expression relative to the blank control, with the GBR experimental group again exhibiting higher OCN expression than the GBR control.

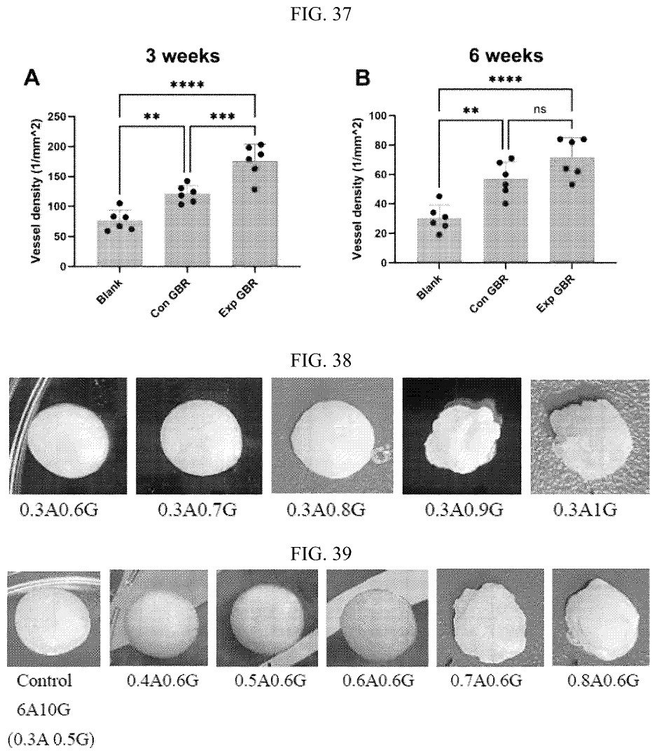

Vascular density, defined as the number of vessels characterized by CD31-positive endothelial cells within the ROI, was also significantly higher in the GBR control and GBR experimental groups compared to the blank control at 3 weeks after grafting (+58.30%, P<0.01; +131.01%, P<0.0001). In addition, vascular density in the GBR experimental group was significantly higher than in the GBR control (+45.9%, P<0.001) (FIGS. 36 and 37). Similarly, at 6 weeks, both the GBR control and GBR experimental groups exhibited significantly increased vascular density, with the GBR experimental group showing a higher vascular density than the GBR control.

### 6. Statistical Analysis

All ex vivo experiments were performed in triplicate. The sample size for the animal experiments (n=5) was determined using G-Power software developed at the Universität Düsseldorf. Based on a preliminary study evaluating the volume of regenerated bone, a statistical power of 90% with a significance level of P<0.05 was adopted. Data are expressed as means and standard deviations. All parameters were analyzed using two-way ANOVA followed by post hoc Tukey's HSD test, and the normality of the data distribution was assessed using the Kolmogorov-Smirnov test. A p-value of less than 0.05 (P<0.05) was considered statistically significant. For histological analysis, comparison between the groups was performed using the non-parametric Mann-Whitney U test. Statistical analyses were conducted using SPSS software (version 16, SPSS Inc.).

What is claimed is:

1. A membrane for guided bone regeneration, comprising:
a hydrogel including alginate and gelatin at a weight ratio of 1:1.0 to 1.6, the hydrogel impregnated with a bone morphogenetic protein (BMP).

2. The membrane of claim 1, wherein the alginate is included at a concentration of 7 to 10% (w/v).

3. The membrane of claim 1, wherein the bone morphogenetic protein is included at a concentration of 0.15 to 0.35% (w/v).

4. The membrane of claim 1, wherein the bone morphogenetic protein is any one selected from the group consisting of BMP-2, BMP-4, BMP-6, BMP-7, and BMP-9.

5. A dual-syringe mixer comprising:
a first syringe filled with a hydrogel preparation powder including alginate and gelatin at a weight ratio of 1:1.0-1.6; and
a second syringe filled with a bone morphogenetic protein (BMP) solution.

6. The dual-syringe mixer according to claim 5, wherein the bone morphogenetic protein is included at a concentration of 0.15 to 0.35% (w/v).

7. The dual-syringe mixer according to claim 5, wherein the bone morphogenetic protein is any one selected from the group consisting of BMP-2, BMP-4, BMP-6, BMP-7, and BMP-9.

8. A method for manufacturing a membrane for guided bone regeneration, the method comprising:
preparing a dispersion by dispersing alginate and gelatin in a buffer at a weight ratio of 1:1.0-1.6;
preparing a mixture by mixing a bone morphogenetic protein (BMP) into the dispersion; and
forming the mixture using a syringe or a 3D printer.

9. The method of claim 8, wherein the forming is performed by injection or 3D printing using a 23 G to 25 G needle.

10. A method for promoting bone formation, the method comprising:
filling a bone defect site with a filler material; and
covering an upper surface of the bone defect site with a membrane for guided bone regeneration to isolate the bone defect site from the outside.

11. The method of claim 10, wherein the isolation of the bone defect site from the outside comprises:
mixing a hydrogel preparation powder and a bone morphogenetic protein (BMP) solution using a dual-syringe mixer, the dual-syringe mixer comprising a first syringe filled with a hydrogel preparation powder including alginate and gelatin at a weight ratio of 1:1.0-1.6 and a second syringe filled with a bone morphogenetic protein (BMP) solution; and
injecting a membrane composition for promoting bone formation into the bone defect of a patient using either the first syringe or the second syringe of the dual-syringe mixer.
